(19) United States
(12) Patent Application Publication
(10) Pub. No.: US 2004/0138412 A1
(43) Pub. Date: Jul. 15, 2004

(54) Extended native chemical ligation

(76) Inventors: Paolo Botti, Piacenza (IT); James A Bradburne, Redwood City, CA (US); Stephen B H Kent, San Francisco, CA (US); Donald W Low, Burlingame, CA (US)

Correspondence Address: LINIAK, BERENATO & WHITE, LLC, 6550 ROCK SPRING DRIVE, SUITE 240, BETHESDA, MD, 20817, US

(21) Appl. No.: 10/333,017

(22) PCT Filed: Sep. 7, 2001
(86) PCT No.: PCT/US01/28172

(51) Int. Cl. C12P 19/34; C07K 7/08

(52) U.S. Cl. 530/324; 530/409

(57) ABSTRACT

The invention is directed to methods and compositions for chemical ligation of components comprising a first component having a carboxythioester, and preferable an α-carboxythioester, moiety and a second component having an N-substituted, and preferably an Nα-substituted, 2 or 3 carbon chain alkyl or aryl thiol to give a ligation product having an N-substituted amide bond at the ligation site. The reactants of the invention are chemoselective, and the alkyl or aryl thiol moiety is removable from the ligation product. Removal of the alkyl or aryl thiol gives a native amide bond at the ligation site. The methods and compositions of the invention are particularly useful for ligation of peptides and polypeptides. The ligation system of the invention is applicable to a wide variety of molecules, and thus can be exploited to generate peptides, polypeptides and other amino acid containing polymers having a native amide bond at the ligation site.

### CROSS REFERENCE TO RELATED APPLICATIONS

This application claims benefit to provisional application U.S. Serial No. 60/231,339, filed Sep. 8, 2000.

### ACKNOWLEDGMENTS

This invention was supported in part by National Institute of Health Postdoctoral Fellowship grant GN190402. The United States government may have certain rights.

### TECHNICAL FIELD

The present invention relates to methods and compositions for extending the technique of native chemical ligation to permit the ligation of a wide range of peptides, polypeptides, other polymers and other molecules via an amide bond.

### BACKGROUND

Chemical ligation involves the formation of a selective covalent linkage between a first chemical component and a second chemical component. Unique, mutually reactive, functional groups present on the first and second components can be used to render the ligation reaction chemoselective. For example, the chemical ligation of peptides and polypeptides involves the chemoselective reaction of peptide or polypeptide segments bearing compatible unique, mutually-reactive, C-terminal and N-terminal amino acid residues. Several different chemistries have been utilized for this purpose, examples of which include native chemical ligation (Dawson, et al., Science (1994) 266:776-779; Kent, et al., WO 96/34878; Kent, et al., WO 98/28434), oxime forming chemical ligation (Rose, et al., J. Amer. Chem. Soc. (1994) 116:30-34), thioester forming ligation (Schnölzer, et al., Science (1992) 256:221-225), thioether forming ligation (Englebretsen, et al., Tet. Letts. (1995) 36(48):8871-8874), hydrazone forming ligation (Gaertner, et al., Bioconj. Chem. (1994) 5(4):333-338), and thiazolidine forming ligation and oxazolidine forming ligation (Zhang, et al., Proc. Natl. Acad. Sci. (1998) 95(16):9184-9189; Tam, et al., WO 95/00846; U.S. Pat. No. 5,589,356).

Of these methods, only the native chemical ligation approach yields a ligation product having a native amide (i.e. peptide) bond at the ligation site. The original native chemical ligation methodology (Dawson et al., supra; and WO 96/34878) has proven a robust methodology for generating a native amide bond at the ligation site. Native chemical ligation involves a chemoselective reaction between a first peptide or polypeptide segment having a C-terminal α-carboxythioester moiety and a second peptide or polypeptide having an N-terminal cysteine residue. A thiol exchange reaction yields an initial thioester-linked intermediate, which spontaneously rearranges to give a native amide bond at the ligation site while regenerating the cysteine side chain thiol. The primary drawback of the original native chemical ligation approach is that it requires an N-terminal cysteine, i.e., it only permits the joining of peptides and polypeptide segments possessing a cysteine at the ligation site.

Notwithstanding this drawback, native chemical ligation of peptides with N-terminal amino acids other than cysteine has been reported (WO98/28434). In this approach, the ligation is performed using a first peptide or polypeptide segment having a C-terminal α-carboxythioester and a second peptide or polypeptide segment having an N-terminal N-{thiol-substituted auxiliary} group represented by the formula HS—CH₂—CH₂—O—NH-[peptide]. Following ligation, the N-{thiol substituted auxiliary} group is removed by cleaving the HS—CH₂—CH₂—O-auxiliary group to generate a native amide bond at the ligation site. One limitation of this method is that the use of a mercaptoethoxy auxiliary group can successfully lead to amide bond formation only at a glycine residue. This produces a ligation product that upon cleavage generates a glycine residue at the position of the N-substituted amino acid of the second peptide or polypeptide segment. As such, this embodiment of the method is only suitable if one desires the ligation product of the reaction to contain a glycine residue at this position, and in any event can be problematic with respect to ligation yields, stability of precursors, and the ability to remove the O-linked auxiliary group. Although other auxiliary groups may be used, for example the HSCH₂CH₂NH-[peptide], without limiting the reaction to ligation at a glycine residue, such auxiliary groups cannot be removed from the ligated product.

Accordingly, what is needed is a broadly applicable and robust chemical ligation system that extends native chemical ligation to a wide variety of different amino acid residues, peptides, polypeptides, polymers and other molecules by means of an effective, readily removable thiol-containing auxiliary group, and that joins such molecules together with a native amide bond at the ligation site. The present invention addresses these and other needs.

### SUMMARY OF THE INVENTION

The invention is directed to methods and compositions related to extended native chemical ligation. The extended native chemical ligation method of the invention comprises: generating an N-substituted amide-linked initial ligation product of the formula:

J1-C(O)—N(C1(R1C)-2-SH)-J2 I

or

J1-C(O)—N(C1(R1)-C2(R2)-C3(R3)-SH)-J2 II

where J1 is a peptide or polypeptide having one or more optionally protected amino acid side chains, or a moiety of such peptide or polypeptide, a polymer, a dye, a suitably functionalized surface, a linker or detectable marker, or any other chemical moiety compatible with chemical peptide synthesis or extended native chemical ligation; R1, R2 and R3 are independently H or an electron donating group conjugated to C1; with the proviso that at least one of R1, R2 and R3 comprises an electron donating group conjugated to C1; and J2 is a peptide or polypeptide having one or more optionally protected amino acid side chains, or a moiety of such peptide or polypeptide, a polymer, a dye, a suitably functionalized surface, a linker or detectable marker; or any other chemical moiety compatible with chemical peptide synthesis or extended native chemical ligation.

The ligation product is produced by the process of ligating a first component comprising a carboxyl thioester of the formula J1-C(O)SR to a second component comprising an acid stable N-substituted 2 or 3 carbon chain amino alkyl or aryl thiol of the formula:

HS—C2-C1(R1)-HN-J2 III

or

HS—C3(R3)-C2(R2)-C1(R1)-HN-J2 IV

where J2, R1, R2, and R3, are as defined above, and then optionally removing the 2 or 3 carbon chain alkyl or aryl thiol from the N-substituted amide-linked ligation product. In a preferred embodiment, such cleavage is facilitated by forming a resonance stabilized cation at C1 under peptide compatible cleavage conditions. The removal of the alkyl or aryl thiol chain from the N generates a final ligation product of the formula:

J1-C(O)—HN-J2 V

where J1, J2, R1, R2, and R3 are as defined above.

The invention also is directed to compositions for effecting such extended native chemical ligation, and to cartridges and kits that comprise them. The compositions comprise a fully protected, partially protected or fully unprotected acid stable N-substituted, and preferably Nα-substituted, 2 or 3 carbon chain amino alkyl or aryl thiol of the formula:

SX2-C2-C1(R1)-X1N—CH(Z2)-C(O)-J2 VI

or

SX2-C3(R3)-C2(R2)-C1(R1)-X1N—CH(Z2)-C(O)-J2 VII

where X1 is H or an amino protecting group; X2 is H or a thiol protecting group; J2, R1, R2 and R3 are as defined above; and Z2 is any chemical moiety (including, without limitation, an amino acid side chain) compatible with chemical peptide synthesis or extended native chemical ligation. The invention also is directed to chiral forms of such compounds of the invention that are substantially free of racemates or diasterioisomers.

The invention is further directed to solution phase and solid phase methods of producing such fully protected, partially protected or fully unprotected N-substituted 2 or 3 carbon chain amino alkyl or aryl thiols. The methods for producing these compounds include halogen-mediated amino alkylation, reductive amination, and preparation of Nα-protected, N-alkylated, S-protected, amino alkyl- or aryl-thiol amino acid precursors compatible with solid phase peptide synthesis methods.

The J1 moiety of the carboxythioester component can comprise any chemical moiety compatible with the carboxythioester and reaction conditions for extended native chemical ligation, and the N-substituted component of the invention can be provided alone or joined to a wide range of chemical moieties, including amino acids, peptides, polypeptides, nucleic acids or other chemical moieties such as dyes, haptens, carbohydrates, lipids, solid support, biocompatible polymers or other polymers and the like. The extended native chemical ligation method of the invention is robust and can be performed in an aqueous system near neutral pH and at a range of temperature conditions. The methods of producing the N-substituted components of the invention also are robust, providing a wide range of synthetic routes to these novel compounds in surprisingly high and pure yields. Nα-protected, N-alkylated, S-protected, amino alkyl- or aryl-thiol amino acid precursors of the invention are particularly useful for rapid automated synthesis using conventional peptide synthesis and other organic synthesis strategies. Moreover, the protected N-substituted components of the invention expand the utility of chemical ligation to multi-component ligation schemes, such as when producing a polypeptide involving multiple ligation strategies, such as a three or more segment ligation scheme or convergent ligation synthesis schemes. For example, the methods and compositions of the present invention permit one to use a first pair of carboxythioester and N-substituted components to synthesize a first portion of a desired molecule, and to use additional pairs of carboxythioester and N-substituted components to synthesize additional portions of the molecule. The ligation products of each such synthesis can then be ligated together (after suitable deprotection and/or modification) to form the desired molecule.

Accordingly, the methods and compositions of the invention greatly expand the scope of native chemical ligation, and the starting, intermediate and final products of the invention find a wide range of uses.

### BRIEF DESCRIPTION OF THE DRAWINGS

FIG. 1 illustrates the present invention by showing its ability to mediate the extended native chemical ligation of peptides; the same schemes could be employed to effect the ligation of any suitable molecule. As shown in the Figure, a first component containing an α-carboxyl thioester of the formula J1-HN—CH(Z1)-αCO—SR, and a second component containing an N-terminal acid stable Nα-substituted 2 carbon chain alkyl or aryl thiol of the formula HS—C2-C1(R1)-NHα-CH(Z2)-C(O)-J2. The components J1 and J2 can be any chemical moiety compatible with the chemoselective ligation reaction, such as a protected or unprotected amino acid, peptide, polypeptide, other polymer, dye, linker and the like. Z1 is any side chain group compatible with the αCO—SR thioester, such as a protected or unprotected side chain of an amino acid. Z2 is any side chain group compatible with an Nα-substituted amino acid, such as a protected or unprotected side chain of an amino acid. R1 is a benzyl moiety (benzyl when referred to in the context of C1, otherwise referred to as phenyl) substituted with an electron-donating group preferably in the ortho or para position relative to C1; or a picolyl (unsubstituted or substituted with hydroxyl or thiol in the ortho or para position relative to C1).

Thiol exchange occurs between the αCOSR thioester component and the amino N-alkyl thiol) component. The exchange generates a thioester-linked intermediate ligation product that after spontaneous rearrangement through a 5-membered ring intermediate generates a first ligation product of the formula J1-HN—CH(Z1)-C(O)—Nα(C1(R1)-C2-SH)—CH(Z2)-C(O)-J2 having a removable Nα-substituted 2 carbon chain alkyl or aryl thiol [HS—C2-C1(R1)-] at the ligation site. The Nα-substituted 2 carbon chain alkyl or aryl thiol [HS—C2-C1(R1)-] at the ligation site is amenable to being removed, under peptide-compatible conditions, to generate a final ligation product of the formula J1-HN—CH(Z1)-CO—NH—CH(Z2)-CO-J2 having a native amide bond at the ligation site.

FIG. 2 illustrates the present invention by showing its ability to mediate the extended native chemical ligation of peptides; the same schemes could be employed to effect the ligation of any suitable molecule. As shown in the Figure, a first component containing α-carboxyl thioester of the formula J1-HN—CH(Z1)-αCO—SR, and a second component containing an acid stable Nα-substituted 3 carbon chain alkyl or aryl thiol of the formula HS—C3(R3)-C2(R2)-C1(R1)-NHα-CH(Z2)-C(O)-J2. The components J1 and J2 can be any chemical moiety compatible with the chemoselective ligation reaction, such as a protected or unprotected amino acid, peptide, polypeptide, other polymer, dye, linker and the like. Z1 is any side chain group compatible with the αCO—SR thioester, such as a protected or unprotected side chain of an amino acid. Z2 is any side chain group compatible with an Nα-substituted amino acid, such as a protected or unprotected side chain of an amino acid. When R1 is other than hydrogen, R2 and R3 are hydrogen, and R1 is a phenyl moiety, unsubstituted or substituted with an electron-donating group in the ortho or para position relative to C1; a picolyl (unsubstituted or substituted with hydroxyl or thiol in the ortho or para position relative to C1); a methanethiol; or a sulfoxymethyl. When R2 and R3 are other than hydrogen, R1 is hydrogen, and R3 and R2 form a benzyl group that is substituted with an electron-donating group in the ortho or para position relative to C1; or a picolyl (unsubstituted or substituted with hydroxyl or thiol in the ortho or para position relative to C1).

Thiol exchange occurs between the COSR thioester component and the amino alkyl thiol component. The exchange generates a thioester-linked intermediate ligation product that after spontaneous rearrangement through a 6-membered ring intermediate generates a first ligation product of the formula J1-HN—CH(Z1)-C(O)—Nα(C1-C2(R2)-C3(R3)-SH)—CH(Z2)-J2 having a removable Nα-substituted 3 carbon chain alkyl or aryl thiol [HS—C3(R3)-C2(R2)-C1(R1)-] at the ligation site. The Nα-substituted 3 carbon chain aryl thiol [HS—C3(R3)-C2(R2)-C1(R1)-] at the ligation site is amenable to being removed, under peptide-compatible conditions, to generate a final ligation product of the formula J1-HN—CH(Z1)-CO—NH—CH(Z2)-CO-J2 having a native amide bond at the ligation site.

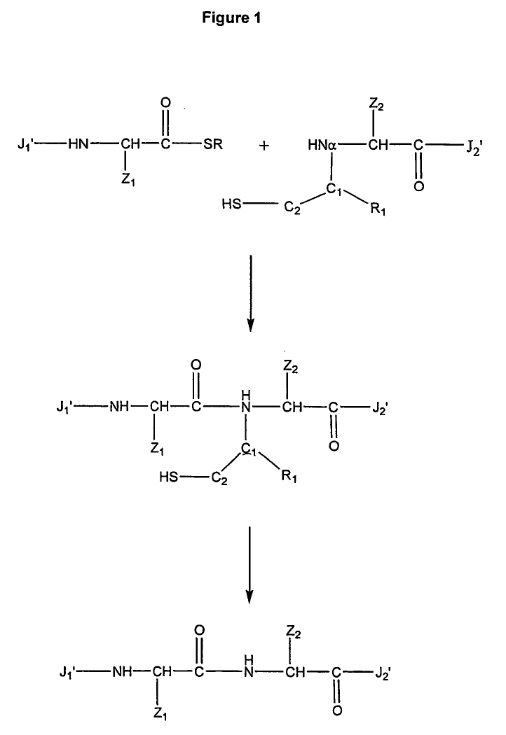

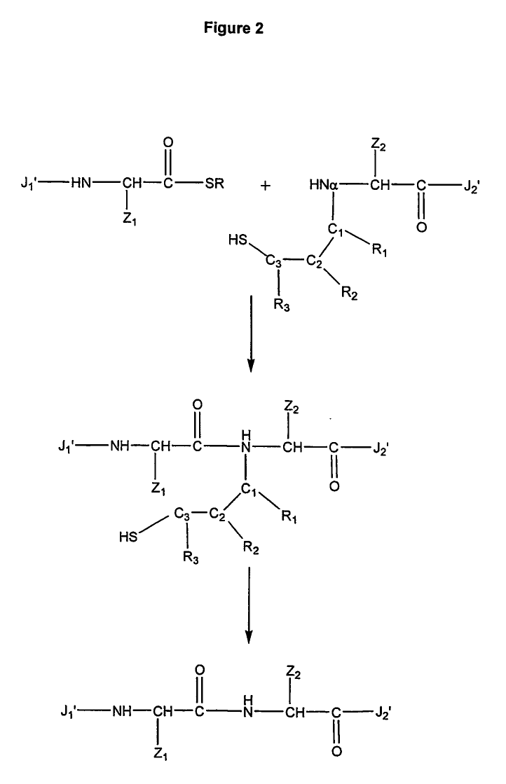

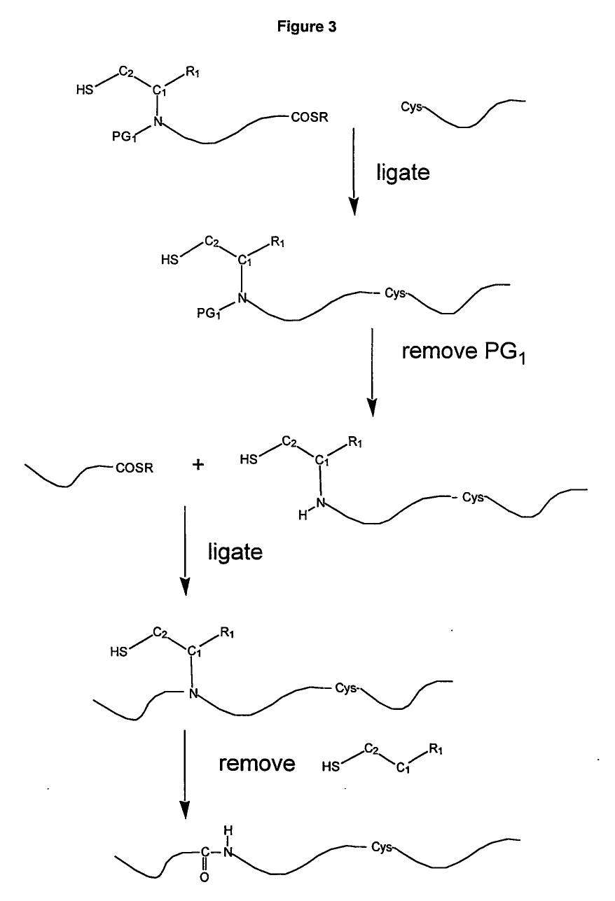

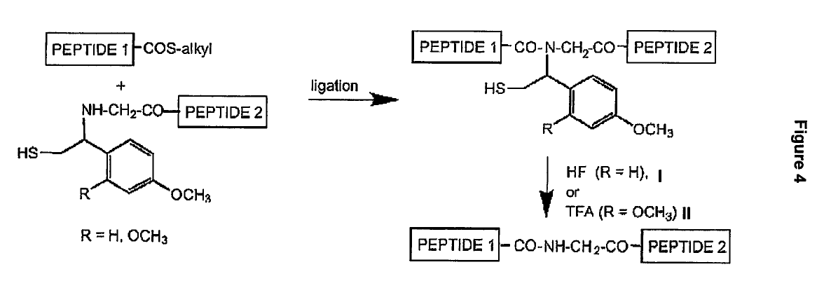

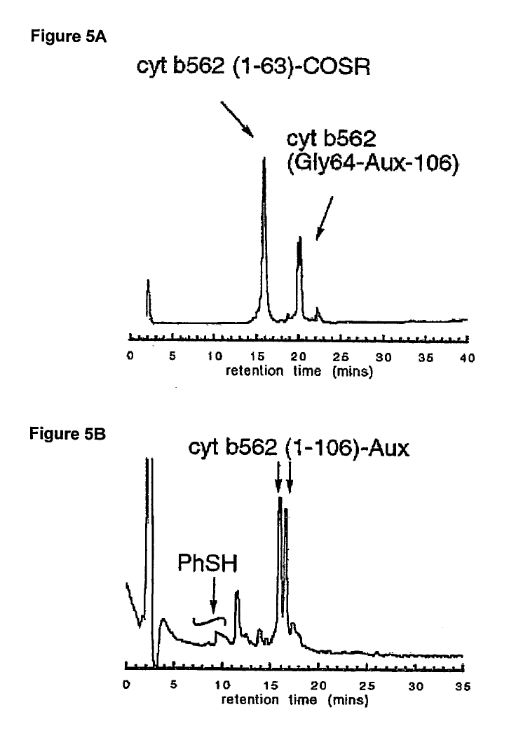

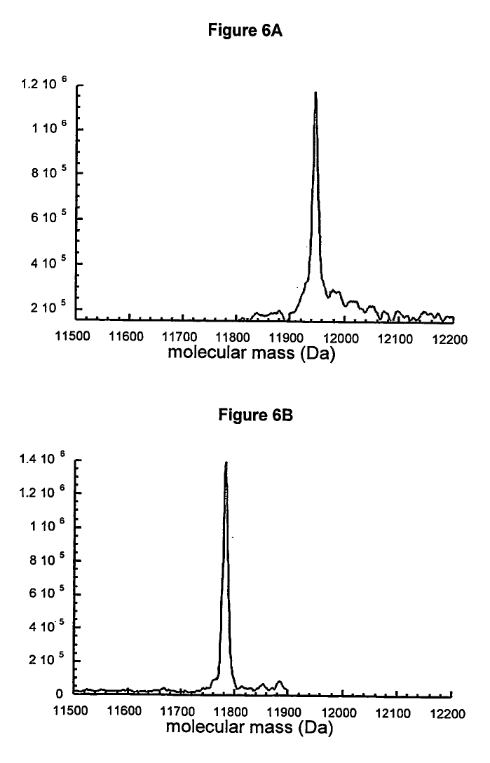

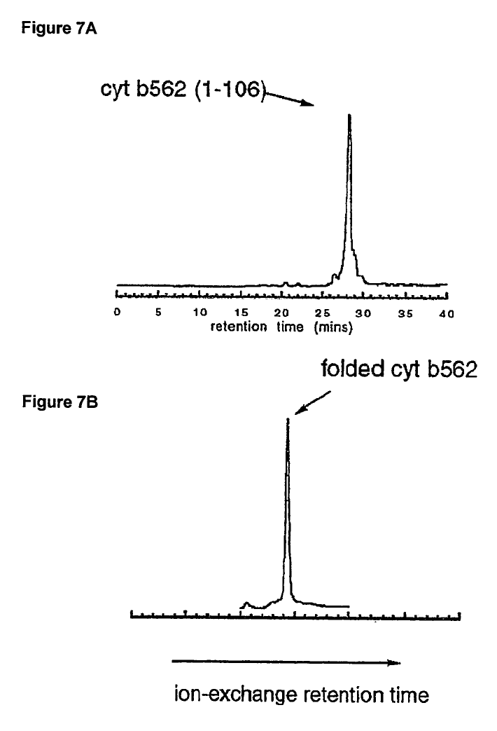

FIG. 3 illustrates a multi-component extended native chemical ligation scheme. A polypeptide α-carboxyl thioester with an Nα-protected N-terminal polypeptide Nα-substituted 2 carbon chain alkyl or aryl thiol of the formula HS—C2-C1(R1)-Nα(PG1)-CH(Z2)-C(O)-J2 as embodied in FIG. 1 is reacted with a peptide that contains an N-terminal Cys residue. R1 is a phenyl, unsubstituted, or substituted with an electron-donating group, preferably in the ortho or para position relative to C1; or a picolyl (unsubstituted or substituted with hydroxyl or thiol in the ortho or para position relative to C1). The protecting group (PG1) may be any suitable protecting group, such as an alkylcarbonyl protecting group (e.g., benzyloxycarbonyl (Z), Boc, Bpoc, Fmoc, etc.), a triphenylmethyl protecting group (Trt), a 2-nitrophenylsulfenyl protecting group (Nps), etc. The protecting group is removed after the first ligation reaction.

A first native chemical ligation reaction is carried out between the polypeptide α-carboxyl thioester with an Nα-protected N-terminal polypeptide Nα-substituted 2 carbon chain alkyl or aryl thiol of the formula HS—C2-C1(R1)-Nα(PG1)-CH(Z2)-C(O)-J2 as embodied in FIG. 1 and the N-terminal Cys-peptide to give a first ligation product of formula: HS—C2-C1(R1)-Nα(PG1)-CH(Z2)-C(O)-Peptide2-Peptide3. The protecting group PG1 is then removed to give the ligation product of formula HS—C2-C1(R1)-Nα(H)—CH(Z2)-C(O)-Peptide2-Peptide3. This species is then reacted with a third, thioester-containing component. Thiol exchange occurs between the COSR thioester component and the amino N{alkyl thiol} component. The exchange generates a thioester-linked intermediate ligation product that after spontaneous rearrangement through a 5-membered ring intermediate generates a second ligation product of the formula Peptide1-C(O)—Nα(C1(R1)-C2-SH)—CH(Z2)-C(O)Peptide2-Cys-Peptide3, having a removable Nα-substituted 2 carbon chain alkyl or aryl thiol [HS—C2-C1(R1)-] at the second ligation site. The Nα-substituted 2 carbon chain alkyl or aryl thiol [HS—C2-C1(R1)-] at the second ligation site is amenable to being removed, under peptide-compatible conditions, to generate a final ligation product of the formula peptide1-C(O)—NαH—CH(Z2)-C(O)Peptide2-Cys-Peptide3, having a native amide bond at the first and second ligation sites.

FIG. 4 illustrates a general ligation strategy employing two different 1-phenyl-2-mercaptoethyl auxiliaries of the invention.

FIGS. 5A and 5B shows analytical High Performance Liquid Chromatography (HPLC) results of a ligation reaction for cytochrome b562 as described in Example 21 using an Nα-1-(4-methoxyphenyl)-2-mercaptoethyl auxiliary. FIG. 5A shows the status of the ligation reaction at time=0. FIG. 5B shows the status of the ligation after the reaction is allowed to proceed overnight. As also shown in FIG. 5B, two ligation products are observed that result from the achiral center at C1 of the Nα-1-(4-methoxyphenol)-2-mercaptoethyl auxiliary.

FIGS. 6A and 6B shows reconstructed electrospray mass spectra (MS) of the ligation product Cytochrome b562 residues 1-106 formed by using extended native chemical ligation with an Nα-{1-(4-methoxyphenyl) 2-mercaptoethano}-modified N-terminal segment. Cytochrome b562 residues 1-63 bearing a C-terminal αthioester was ligated with Cytochrome b562 residues 64-106 bearing an N-terminal Nα-{1-(4-methoxyphenyl) 2-mercaptoethano} glycine. FIG. 6A shows MS reconstruct of the initial ligation product that includes a removable Nα-{1-(4-methoxyphenyl) 2-mercaptoethano} group at the ligation site. FIG. 6B shows a MS reconstruct of ligation product following hydrogen fluoride (HF) treatment to remove the Nα-{1-(4-Methoxyphenyl) 2-mercaptoethano} group to generate a native amide bond at the ligation site. The observed masses were 11948±1 Da (before HF treatment) and 11781±1 Da (after HF treatment), i.e. a loss of 167±2 Da, in good agreement with the 166 Da loss expected for removal of the 1-(4methoxyphenyl) 2-mercaptoethano auxiliary group.

FIGS. 7A and 7B illustrate a representative analytical HPLC of linear cytochrome b562 material (FIG. 7A) depicted in FIG. 6B, and an ion exchange chromatogram (FIG. 7B) of the material following folding.

### DESCRIPTION OF SPECIFIC EMBODIMENTS

The invention is directed to methods and compositions related to extended native chemical ligation. In general, the method involves ligating a first component comprising a carboxyl thioester, and more preferably, an α-carboxyl thioester with a second component comprising an acid stable N-substituted, and preferably, Nα-substituted, 2 or 3 carbon chain amino alkyl or aryl thiol. Chemoselective reaction between the carboxythioester of the first component and the thiol of the N-substituted 2 or 3 carbon chain alkyl or aryl thiol of the second component proceeds through a thioester-linked intermediate, and resolves into an initial ligation product. More specifically, the thiol exchange occurring between the COSR thioester component and the amino alkyl thiol component generates a thioester-linked intermediate ligation product that after spontaneous rearrangement through a 5-membered or 6-membered ring intermediate generates an amide-linked first ligation product of the formula:

J1-C(O)—N(C1(R1)-C2-SH)-J2 I

or

J1-C(O)—N(C1(R1)-C2(R2)-C3(R3)-SH)-J2 II

where J1, J2, R1, R2 and R3 are as defined above.

The N-substituted 2 or 3 carbon chain alkyl or aryl thiol [HS—C2-C1(R1)-] or [HS—(C3(R3)-C2(R2)-C1(R1)-] at the ligation site is amenable to being removed, under peptide-compatible conditions, without damage to the product, to generate a final ligation product of the formula:

J1-C(O)—HN-J2 V

where J1, J2, R1, R2, and R3 are as defined above. The final ligation product has a native amide bond at the ligation site

More particularly, the extended native chemical ligation method of the invention comprises chemical ligation of: (i) a first component comprising an α-carboxyl thioester of the formula J1-C(O)SR and (ii) a second component comprising an acid stable N-substituted 2 or 3 carbon chain amino alkyl or aryl thiol of the formula:

J1-C(O)—N(C1(R1)-C2-SH)-J2 I

or

J1-C(O)—N(C1(R1)-C2(R2)-C3(R3)-SH)-J2 II

where J1, J2, R1, R2, and R3 are as defined above.

The R1, R2 and R3 groups are selected to facilitate cleavage of the N—C1 bond under peptide compatible cleavage conditions. For example, electron donating groups, particularly if conjugated to C1, can be used to form a resonance stabilized cation at C1 that facilitates cleavage. The chemical ligation reaction preferably includes as an excipient a thiol catalyst, and is carried out around neutral pH conditions in aqueous or mixed organic-aqueous conditions. Chemical ligation of the first and second components may proceed through a five or six member ring that undergoes spontaneous rearrangement to yield an N-substituted amide linked ligation product. Where the first and second components are peptides or polypeptides, the N-substituted amide linked ligation product has the formula:

J1-C(O)—Nα(C1(R1)-C2-SH)—CH(Z2)-C(O)-J2 VIII

or

J1-C(O)—Nα(C1(R1)-C2(R2)-C3(R3)-SH)—CH(Z2)-C(O)-J2 IX

where J1, J2 and R1, R2, R3 and Z2 are as defined above

The conjugated electron donating groups R1, R2 or R3 of the N-substituted amide bonded ligation product facilitate cleavage of the N—C1 bond and removal of the 2 or 3 carbon chain alkyl or aryl thiol from the N-substituted amide-linked ligation product. Removal of the alkyl or aryl thiol chain of the N under peptide-compatible cleavage conditions generates a ligation product having a native amide bond at the ligation site. If the first and second components were peptides or polypeptides, the ligation product will have the formula:

J1-CONαH—CH(Z2)-C(O)-J2 X

The present invention provides multiple advantages over previous chemical ligation approaches. Several such advantages relate to the finely tuned nature of the N-substituted 2 or 3 carbon chain alkyl or aryl thiol component of the present invention. First, the unligated N-substituted component is stable to acidic conditions, which permits its robust synthesis and storage. Second, it selectively reacts with the carboxythioester component to generate an initial ligation product having an N-substituted amide bond at the ligation site. Third, the regenerated alkyl or aryl thiol moiety at the Nα position of the ligation site of the initial ligation product can be selectively removed under conditions fully compatible with unprotected, partially protected or fully protected peptides, polypeptides or other moieties, i.e., the alkyl or aryl thiol moiety can be removed without damaging the desired ligation product. The selective cleavage reaction can be readily performed under standard peptide-compatible cleavage conditions such as acidic, photolytic, or reductive conditions, depending on the particular N-substituted alkyl or aryl thiol moiety chosen for ligation. Thus, another advantage of the invention is that one or more groups on remaining portions of the ligation components, if present, can be unprotected, partially protected or fully protected depending on the intended end use. Moreover, given the chemoselective nature and solubility properties of the carboxyl thioester and N-substituted 2 or 3 carbon chain alkyl or aryl thiol, the ligation reaction can be carried out rapidly and cleanly to give high product yields at around pH 7 under aqueous conditions at around room temperature. This makes the invention particularly flexible for ligating partially or fully unprotected peptides, polypeptides or other polymers under mild conditions.

For a peptide component that comprises the N-substituted 2 carbon chain alkyl or aryl thiol component of the invention, this compound has the formula:

HS—C2-C1(R1)-NHα-CH(Z2)-C(O)-J2 XI

as depicted below in Table I. J2 and R2 are as described above; Z2 is any side chain group compatible with an N-substituted amino acid, such as a side chain of an amino acid. R1 is preferably a phenyl group substituted with an electron-donating group in the ortho or para position relative to C1; or a picolyl group (unsubstituted or substituted with hydroxyl or thiol in the ortho or para position relative to C1).

TABLE I
Formula I

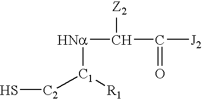

R1 Substituent Groups for Formula I (C1 included for reference)

| | | |
|---|---|---|
| 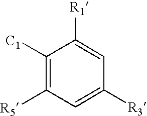 | 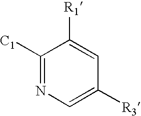 | 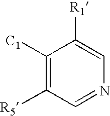 |

Positioning of the phenyl and picolyl electron-donating substituents R1′, R3′ and R5′ in the ortho or para positions is necessary to maintain electronic conjugation to the C1 carbon to enhance cleavage of the N—C1 bond following ligation. Preferred electron-donating groups for R1′, R3′ and R5′ include strong electron-donating groups such as methoxy (—OCH3), thiol (—SH), hydroxyl (—OH), methylthio (—SCH3), and moderate electron-donators such as methyl (—CH3), ethyl (—CH2-CH3), propyl (—CH2-CH2-CH3), isopropyl (—CH2(CH3)3). Provided that any or all of R1′, R3′ and R5′ maybe H. A general observation is that the strong electron-donating groups enhance the sensitivity of the 2-carbon chain alkyl or aryl thiol to cleavage following ligation. When a single electron-donating group is present as a R1′, R3′ or R5′ substituent, the ligation reaction may proceed at a faster rate, whereas cleavage is slower or requires more stringent cleavage conditions. When two or more electron-donating groups are present as a R1′, R3′ or R5′ substituent, the ligation reaction may be slower, whereas cleavage is faster or requires less stringent cleavage conditions._Thus a particular electron-donating group can be selected accordingly.

Another embodiment of the invention relates to the N-substituted 2 carbon chain compounds, which include a thiol as a substituent of R1 in the R1′ and R5′ positions. In addition to being an electron donating group conjugated to C1, introduction of a thiol at one or both of these locations enables the compounds to ligate through a 6-member ring mediated through the R1 group (as well as through a 5-member ring by the Nα-2 carbon chain alkyl thiol). It also increases the local concentration of available thiols for reacting with the α-carboxy thioester, and provides for additional conformations in terms of structural constraints that can improve ligation.

Referring to the Nα-substituted 3 carbon chain alkyl or aryl thiol component of the invention, this compound has the formula HS—C3(R3)-C2(R2)-C1(R1)-NHα-CH(Z2)-C(O)-J2, which is depicted below in Table II.

TABLE II
Formula II

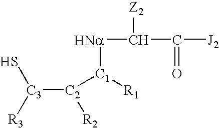

R1, R2 and R3 Substituents (C1 included for reference)

| | | |
|---|---|---|
| 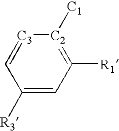 | 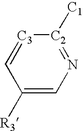 | 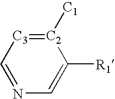 |
| 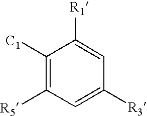 | 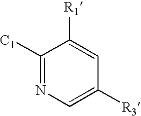 | 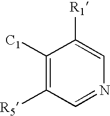 |

As described above, J2 can be any chemical moiety compatible with the chemical peptide synthesis or extended native chemical ligation, Z2 is any side chain group compatible with an N-substituted amino acid, such as a side chain of an amino acid. When R1 is other than hydrogen, R2 and R3 are hydrogen, and R1 is a phenyl moiety, unsubstituted, or more preferably, substituted with an electron-donating group in the ortho or para position relative to C1; or a picolyl (unsubstituted or substituted with hydroxyl or thiol in the ortho or para position relative to C1). When R2 and R3 are other than hydrogen, R1 is hydrogen, and R3 and R2 form a benzyl group that is substituted with an electron-donating group in the ortho or para position relative to C1; or a picolyl (unsubstituted or substituted with hydroxyl or thiol in the ortho or para position relative to C1).

As with the N-substituted 2 carbon chain compounds, positioning of the phenyl and picolyl electron-donating substituents R1′, R3′ and R5′ in the ortho or para positions is necessary to maintain electronic conjugation to the C1 carbon for robust cleavage of the Nα-C1 bond following ligation. However, when R2 and R3 form a benzyl group with C2 and C3, at least one of R1′ and R3′ comprises a strong electron donating group, where R1′ or R3′ is selected from methoxy (—OCH3), thiol (—SH), hydroxyl (—OH), and thiomethyl (—SCH3). For the N-substituted 3 carbon chain thiols in which R2 and R3 are hydrogens, R1 comprises a phenyl or picolyl group in which R1′, R3′ and R5′ include either strong or moderate electron-donating groups, or a combination thereof. As with the N-substituted 2 carbon chain alkyl or aryl thiols, the strong electron-donating groups enhance the sensitivity of the 3 carbon chain alkyl or aryl thiol to cleavage following ligation. Thus a particular electron-donating group or combination thereof can be selected accordingly.

Similar to the N-substituted 2 carbon chain compounds, the N-substituted 3 carbon chain compounds of the present invention may include a thiol as a substituent of R1 in the R1′ and R5′ positions when available for substitution in a construct. Here again the electron-donating thiol group is conjugated to C1 and its introduction at these locations enables the compounds to have two routes for the 6-member ring forming ligation event. It also increases the local concentration of available thiols for reacting with the α-carboxy thioester, and provides for additional conformations in terms of structural constraints that can improve ligation.

Synthesis of the N-terminal N-substituted 2 or 3 carbon chain alkyl or aryl thiol amino acids of the invention can carried out as described herein, for example, in Scheme I and Scheme II, the Examples, and in accordance with standard organic chemistry techniques known in the art. See, e.g., “Advanced Organic Chemistry, Reactions, Mechanisms, and Structure,” 4^{th} Edition, J. March (Ed.), John Wiley & Sons, New York, N.Y., 1992; “Comprehensive Organic Transformations, A Guide to Functional Group Preparations,” R. Larock (Ed.), VCH Publishers, New York, N.Y., 1989. They may be synthesized in solution, by polymer-supported synthesis, or a combination thereof. The preferred approach employs N alpha protected N alkylated S-protected amino alkyl- or aryl-thiol amino acid precursors. The reagents utilized for synthesis can be obtained from any number of commercial sources. Also, it will be well understood that the starting components and various intermediates, such as the individual amino acid derivatives can be stored for later use, provided in kits and the like.

In preparing the N-terminal Nα-substituted 2 or 3 carbon chain alkyl or aryl thiol amino acids of the invention, protecting group strategies are employed. The preferred protecting groups (PG) utilized in the various synthesis strategies in general are compatible with Solid Phase Peptide Synthesis (“SPPS”). In some instances, it also is necessary to utilize orthogonal protecting groups that are removable under different conditions. Many such protecting groups are known and suitable for this purpose (See, e.g., “Protecting Groups in Organic Synthesis”, 3rd Edition, T. W. Greene and P. G. M. Wuts, Eds., John Wiley & Sons, Inc., 1999; NovaBiochem Catalog 2000; “Synthetic Peptides, A User's Guide,” G. A. Grant, Ed., W.H. Freeman & Company, New York, N.Y., 1992; “Advanced Chemtech Handbook of Combinatorial & Solid Phase Organic Chemistry,” W. D. Bennet, J. W. Christensen, L. K. Hamaker, M. L. Peterson, M. R. Rhodes, and H. H. Saneii, Eds., Advanced Chemtech, 1998; “Principles of Peptide Synthesis, 2nd ed.,” M. Bodanszky, Ed., Springer-Verlag, 1993; “The Practice of Peptide Synthesis, 2nd ed.,” M. Bodanszky and A. Bodanszky, Eds., Springer-Verlag, 1994; and “Protecting Groups,” P. J. Kocienski, Ed., Georg Thieme Verlag, Stuttgart, Germany, 1994). Examples include benzyloxycarbonyl (Z), Boc, Bpoc, Trt, Nps, FmocCl-Z, Br-Z; NSC; MSC, Dde, etc. For sulfur moieties, examples of suitable protecting groups include, but are not limited to, benzyl, 4-methylbenzyl, 4-methoxybenzyl, trityl, Acm, TACAM, xanthyl, disulfide derivatives, picolyl, and phenacyl.

More particularly, the Nα-substituted 2 or 3 carbon chain alkyl or aryl thiols can be prepared in accordance with Scheme I (Solid-Phase preparation of the Nα-substituted precursor), Scheme II (Solution-Phase preparation of the Nα-substituted precursor). In Scheme I, Nα-substituted 2 or 3 carbon chain alkyl or aryl thiols are assembled directly on the solid phase using standard methods of polymer-supported organic synthesis, while the Nα-protected, N-alkylated, S-protected, aminoalkyl or arylthiol amino acid precursor of Scheme II are coupled to the resin using standard coupling protocols. In Scheme I, X is a halogen, R1 and R2 are as described above and can be preformed as protected or unprotected moieties or elaborated on-resin, and J2 is preferably attached to halogen as X—CH(R)-J2-Resin, where R is hydrogen or other side chain. It will be appreciated that J2 can be a variety of groups, for example where halogen X and J2-Resin are separated by more than one carbon, such as in synthesis of beta or gamma amino acids or similar molecules. Where glyoxalic moiety (HC(O)—C(O)-J2-Resin) is employed, resulting side chain R is hydrogen. In Scheme II, X is a halogen, R1 and R2 are as described above and can be preformed as protected or unprotected moieties or elaborated in solution or on-resin, and where R is hydrogen or other side chain. Where glyoxalic acid moiety (HC(O)—C(O)—OH) is employed, the resulting side chain R is hydrogen. As noted above, it will be appreciated that Schemes I and II can be applied in the synthesis of the 3 carbon chain alkyl or aryl thiols. Where racemic or diastereomeric products are produced, it may be necessary to separate these by standard methods before use in extended native chemical ligation.

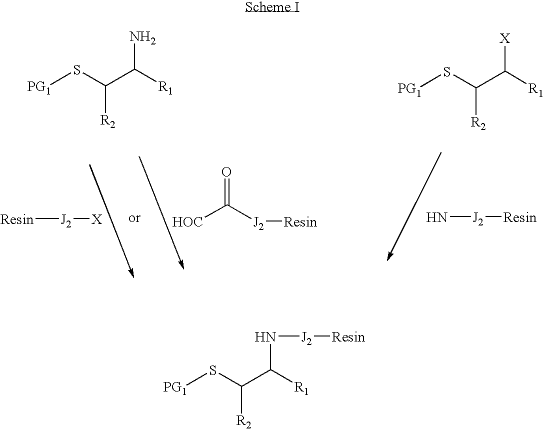

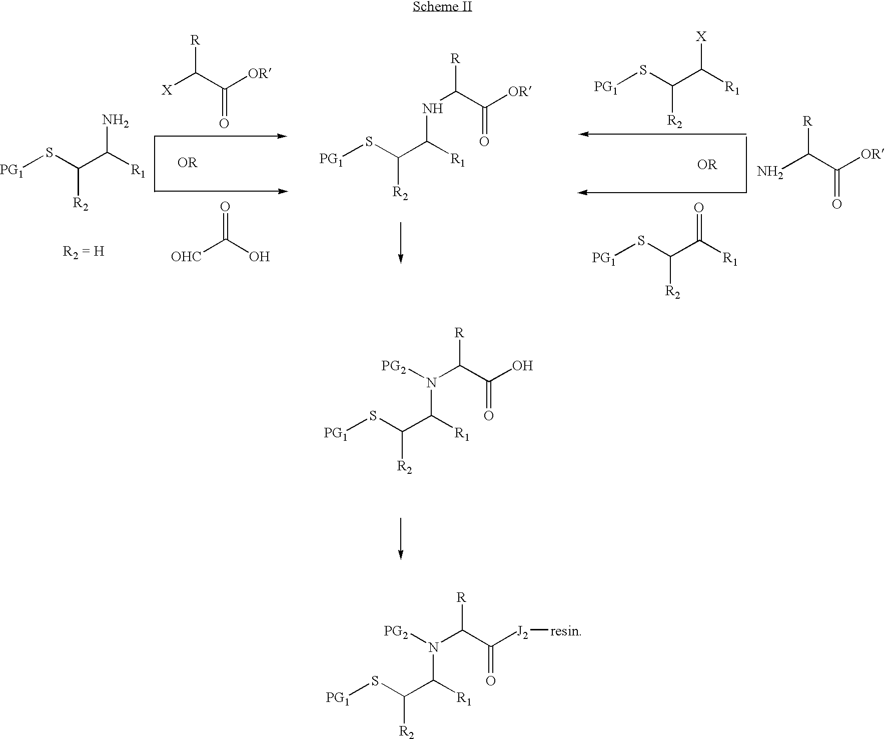

Referring to the carboxy thioester moiety of the first component utilized for the extended native chemical ligation method of the invention, this component has the formula J1-CO—SR. The more preferred carboxy thioester component comprises an α-carboxyl thioester amino acid of the formula J1-NH—C(Z1)-CO—SR. The group J1 can be any chemical moiety compatible with the chemoselective ligation reaction, such as a protected or unprotected amino acid, peptide, polypeptide, other polymer, dye, linker and the like. Z1 is any side chain group compatible with the αCO—SR thioester, such as a side chain of an amino acid. R is any group compatible with the thioester group, including, but not limited to, aryl, benzyl, and alkyl groups. Examples of R include 3-carboxy-4-nitrophenyl thioesters, benzyl thioesters, and mercaptopropionic acid leucine thioesters (See, e.g., Dawson et al., Science (1994) 266:776-779; Canne et al. Tetrahedron Lett (1995) 36:1217-1220; Kent, et al., WO 96/34878; Kent, et al., WO 98/28434; Ingenito et al., JACS (1999) 121(49):11369-11374; and Hackeng et al., Proc. Natl. Acad. Sci. U.S.A. (1999) 96:10068-10073). Other examples include dithiothreitol, or alkyl or aryl thioesters, which can be produced by intein-mediated biological techniques, which also are well known (See, e.g., Chong et al., Gene (1997) 192:277-281; Chong et al., Nucl. Acids Res. (1998) 26:5109-5115; Evans et al., Protein Science (1998) 7:2256-2264; and Cotton et al., Chemistry & Biology (1999) 6(9):247-256).

The α-carboxythioesters can be generated by chemical or biological methods following standard techniques known in the art, such as those described herein, including the Examples. For chemical synthesis, α-carboxythioester peptides can be synthesized in solution or from thioester-generating resins, which techniques are well known (See, e.g., Dawson et al., supra; Canne et al., supra; Hackeng et al., supra, Hojo H, Aimoto, S. (1991) Bull Chem Soc Jpn 64:111-117). For instance, chemically synthesized thioester peptides can be made from the corresponding peptide α-thioacids, which in turn, can be synthesized on a thioester-resin or in solution, although the resin approach is preferred. The peptide-α-thioacids can be converted to the corresponding 3-carboxy-4-nitrophenyl thioesters, to the corresponding benzyl ester, or to any of a variety of alkyl thioesters. All of these thioesters provide satisfactory leaving groups for the ligation reactions, with the 3-carboxy-4-nitrophenyl thioesters demonstrating a somewhat faster reaction rate than the corresponding benzyl thioesters, which in turn may be more reactive than the alkyl thioesters. As another example, a trityl-associated mercaptoproprionic acid leucine thioester-generating resin can be utilized for constructing C-terminal thioesters (Hackeng et al., supra). C-terminal thioester synthesis also can be accomplished using a 3-carboxypropanesulfonamide safety-catch linker by activation with diazomethane or iodoacetonitrile followed by displacement with a suitable thiol (Ingenito et al., supra; Shin et al., (1999) J. Am. Chem. Soc., 121, 11684-11689).

Peptide or polypeptide C-terminal α-carboxythioesters also can be made using biological processes. For instance, intein expression systems, with or without labels such as affinity tags can be utilized to exploit the inducible self-cleavage activity of an “intein” protein-splicing element in the presence of a suitable thiol to generate a C-terminal thioester peptide or polypeptide segment. In particular, the intein undergoes specific self-cleavage in the presence of thiols such as DTT, β-mercaptoethanol, β-mercaptoethanesulfonic acid, or cysteine, which generates a peptide segment bearing a C-terminal thioester. See, e.g., Chong et al., (1997) supra; Chong et al., (1998) supra; Evans et al., supra; and Cotton et al., supra.

Ligation of the N-substituted 2 or 3 carbon chain alkyl or aryl thiol components of the invention with the first carboxythioester component generates a ligation product having an N-substituted amide bond at the ligation site, as depicted in FIGS. 1, 2 and 3. The ligation conditions of the reaction are chosen to maintain the selective reactivity of the thioester with the N-substituted 2 or 3 carbon chain alkyl or aryl thiol moiety. In a preferred embodiment, the ligation reaction is carried out in a buffer solution having pH 6-8, with the preferred pH range being 6.5-7.5. The buffer solution may be aqueous, organic or a mixture thereof. The ligation reaction also may include one or more catalysts and/or one or more reducing agents, lipids, detergents, other denaturants or solubilizing reagents and the like. Examples of preferred catalysts are thiol and phosphine containing moieties, such as thiophenol, benzylmercaptan, TCEP and alkyl phosphines. Examples of denaturing and/or solubilizing agents include guanidinium, urea in water or organic solvents such as TFE, HFIP, DMF, NMP, acetonitrile admixed with water, or with guanidinium and urea in water. The temperature also may be utilized to regulate the rate of the ligation reaction, which is usually between 5° C. and 55° C., with the preferred temperature being between 15° C. and 40° C. As an example, the ligation reactions proceed well in a reaction system having 2% thiophenol in 6M guanidinium at a pH between 6.8 and 7.8.

For the N-substituted 2 carbon chain alkyl or aryl thiols, the ligation event results from a thiol exchange that occurs between the COSR thioester component and the amino alkyl thiol component. The exchange generates a thioester-linked intermediate ligation product that after spontaneous rearrangement through a 5-membered ring intermediate generates a first ligation product of the formula J1-HN—CH(Z1)-C(O)—Nα(C1(R1)-C2-SH)—CH(Z2)-J2 having a removable N-substituted 2 carbon chain alkyl or aryl thiol [HS—C2-C1(R1)-] at the ligation site, where the substituents are as defined above. The N-substituted 2 carbon chain alkyl or aryl thiol [HS—C2-C1(R1)-] at the ligation site is amenable to being removed, under peptide-compatible conditions, to generate a final ligation product of the formula J1-HN—CH(Z1)-CO—NH—CH(Z2)-CO-J2 having a native amide bond at the ligation site.

For the N-substituted 3 carbon chain aryl or alkyl thiols, the thiol exchange between the COSR thioester component and the amino alkyl thiol component generates a thioester-linked intermediate ligation product that after spontaneous rearrangement through a 6-membered ring intermediate generates a first ligation product of the formula J1-HN—CH(Z1)-C(O)—Nα(C1-C2(R2)-C3(R3)-SH)—CH(Z2)-J2 having a removable N-substituted 3 carbon chain alkyl or aryl thiol [HS—C3(R3)-C2(R2)-C1(R1)-] at the ligation site. The N-substituted 3 carbon chain aryl thiol [HS—C3(R3)-C2(R2)-C1(R1)-] at the ligation site is amenable to being removed, under peptide-compatible conditions, to generate a final ligation product of the formula J1-HN—CH(Z1)-CO—NH—CH(Z2)-CO-J2 having a native amide bond at the ligation site.

Removal of the N-substituted alkyl or aryl thiol group is preferably performed in acidic conditions to facilitate cleavage of the N—C1 bond, yielding a stabilized, unsubstituted amide bond at the ligation site. By “peptide-compatible cleavage conditions” is intended physical-chemical conditions compatible with peptides and suitable for cleavage of the N-linked alkyl or aryl thiol moiety from the ligation product. Peptide-compatible cleavage conditions in general are selected depending on the Nα-alkyl or aryl thiol moiety employed, which can be readily deduced through routine and well known approaches (See, e.g., “Protecting Groups in Organic Synthesis”, 3rd Edition, T. W. Greene and P. G. M. Wuts, Eds., John Wiley & Sons, Inc., 1999; NovaBiochem Catalog 2000; “Synthetic Peptides, A User's Guide,” G. A. Grant, Ed., W.H. Freeman & Company, New York, N.Y., 1992; “Advanced Chemtech Handbook of Combinatorial & Solid Phase Organic Chemistry,” W. D. Bennet, J. W. Christensen, L. K. Hamaker, M. L. Peterson, M. R. Rhodes, and H. H. Saneii, Eds., Advanced Chemtech, 1998; “Principles of Peptide Synthesis, 2nd ed.,” M. Bodanszky, Ed., Springer-Verlag, 1993; “The Practice of Peptide Synthesis, 2nd ed.,” M. Bodanszky and A. Bodanszky, Eds., Springer-Verlag, 1994; and “Protecting Groups,” P. J. Kocienski, Ed., Georg Thieme Verlag, Stuttgart, Germany, 1994).

For example, where the R1′, R2′ or R3′ substituents comprises a methoxy, hydroxyl, thiol or thiomethyl, methyl and the like, the more universal method for removal involves acidic cleavage conditions typical for peptide synthesis chemistries. This includes cleavage of the N—C1 bond under strong acidic conditions or water-acidic conditions, with or without reducing reagents and/or scavenger systems (e.g., acid such as anhydrous hydrogen fluoride (HF), triflouroacetic acid (TFA), or trifluoromethanesulfonic acid (TFMSA) and the like). More specific acidic cleavage systems can be chosen to optimize cleavage of the Nα-C1 bond to remove the aryl or alkyl thiol moiety for a given construct. Such conditions are well known and compatible with maintaining the integrity of peptides. Another method for cleavage involves the inclusion of a thiol scavenger where tryptophans are present in a peptide or polypeptide sequence to avoid reaction of the tryptophan side chain with the liberated aryl or alkyl thiol moiety. Examples of thiol scavengers include ethanediol, cysteine, beta-mercaptoethanol and thiocresol. Accordingly, another embodiment of the invention is the addition of a thiol scavenger when cleaving the N—C1 bond to remove the aryl or alkyl thiol moiety.

Other specialized cleavage conditions include light or reductive-cleavage conditions when the picolyl group is the substituent. As an example, when the R1, or R2 and R3 substituents comprise a picolyl moiety, photolysis (e.g., ultraviolet light), zinc/acetic acid or electrolytic reduction may be used for cleavage following standard protocols. Where R1 of the N-substituted 2 carbon chain thiol comprises a thiomethane at R1, then mercury(II) or HF cleavages can be used. The cleavage system also can be used for simultaneous cleavage from a solid support and/or as a deprotection reagent when the first or second ligation components comprise other protecting groups. For instance, N-picolyl groups can be removed by dissolving the polypeptide in a 10% acetic acid/water solution, with activated zinc (˜0.5 g/ml). Thiomethane groups, such as 2-mercapto, 1-methylsulfinylethane groups (HS—C2-C1(S(O)—CH3)-Nα), can be removed after ligation by reduction and mercuric, mercaptan-mediated cleavage. As an example, the methylsulfinylethane group can be removed by dissolving the polypeptide in an aqueous 3% acetic acid solution containing N-methylmercaptoacetamide (MMA) (e.g., 1 mg polypeptide in 0.5 ml of acetic acid/water and 0.05 ml of MMA), for reduction to the thiomethane form, followed by freezing and lyophilization of the mixture after overnight reaction. The reduced auxiliary can then be removed in an aqueous solution of 3% acetic acid containing mercury acetate (Hg(OAC)₂) (e.g., 0.5 ml of acetic acid in water and 10 mg of Hg(OAC)₂ for about 1 hour), followed by addition of beta-mercaptoethanol (e.g., 0.2 ml beta-mercaptoethanol). Products can then be purified by standard methods, such as reverse phase HPLC (RPHPLC).

As can be appreciated, one or more catalysts and/or excipients may also be utilized in the cleavage system, such as one or more scavengers, detergents, solvents, metals and the like. In general, selection of specific scavengers depends upon the amino acids present. For instance, the presence of scavengers can be used to suppress the damaging effect that the carbonium ions, produced during cleavage, can have on certain amino acids (e.g., Met, Cys, Trp, and Tyr). Other additives like detergents, polymers, salts, organic solvents and the like also may be employed to improve cleavage by modulating solubility. Catalysts or other chemicals that modulate the redox system also can be advantageous. It also will be readily apparent that a variety of other physical-chemical conditions such as buffer systems, pH and temperature can be routinely adjusted to optimize a given cleavage system.

The present invention also provides protected forms of the Nα-substituted 2 or 3 carbon chain alkyl or aryl thiols of the invention. These compounds are especially useful for automated peptide synthesis and orthogonal and convergent ligation strategies. These compositions comprise a fully protected, partially protected or fully unprotected acid stable Nα-substituted 2 or 3 carbon chain amino alkyl or aryl thiol of the formula (PG2)S—C2-C1(R1)-Nα(PG1)-CH(Z2)-C(O)-J2 or (PG2)S—C3(R3)-C2(R2)-C1(R1)-Nα(PG1)-CH(Z2)-C(O)-J2, which are depicted below in Table III and Table IV. In particular, one or more of R1, R2 and R3 comprises an electron donating group conjugated to C1 that, following conversion of the Nα-substituted amino alkyl or aryl thiol to an Nα-substituted amide alkyl or aryl thiol, is capable of forming a resonance stabilized cation at C1 that facilitates cleavage of the Nα-C1 bond under peptide compatible cleavage conditions. PG1 and PG2 are protecting groups that are present individually or in combination or are absent and can be the same or different, where Z2 is any chemical moiety compatible with chemical peptide synthesis or extended native chemical ligation, and where J2 is any chemical moiety compatible with chemical peptide synthesis or extended native chemical ligation.

PG1 (or X1) is a group for protecting the amine. PG2 (or X2) is a group for protecting the thiol. Many such protecting groups are known and suitable for this purpose (See, e.g., “Protecting Groups in Organic Synthesis”, 3rd Edition, T. W. Greene and P. G. M. Wuts, Eds., John Wiley & Sons, Inc., 1999; NovaBiochem Catalog 2000; “Synthetic Peptides, A User's Guide,” G. A. Grant, Ed., W.H. Freeman & Company, New York, N.Y., 1992; “Advanced Chemtech Handbook of Combinatorial & Solid Phase Organic Chemistry,” W. D. Bennet, J. W. Christensen, L. K. Hamaker, M. L. Peterson, M. R. Rhodes, and H. H. Saneii, Eds., Advanced Chemtech, 1998; “Principles of Peptide Synthesis, 2nd ed.,” M. Bodanszky, Ed., Springer-Verlag, 1993; “The Practice of Peptide Synthesis, 2nd ed.,” M. Bodanszky and A. Bodanszky, Eds., Springer-Verlag, 1994; and “Protecting Groups,” P. J. Kocienski, Ed., Georg Thieme Verlag, Stuttgart, Germany, 1994).

TABLE III

| | |
|---|---|
| 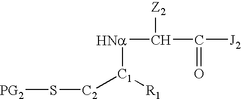 | 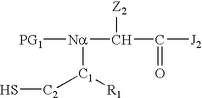 |

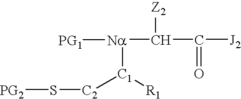

Examples of preferred protecting groups for PG1 and X1 include, but are not limited to [Boc(t-Butylcarbamate), Troc(2,2,2,-Trichloroethylcarbamate), Fmoc(9-Fluorenylmethylcarbamate), Br-Z or Cl-Z(Br- or Cl-Benzylcarbamate), Dde(4,4,-dimethyl-2,6-dioxocycloex1-ylidene), MsZ(4-Methylsulfinylbenzylcarbamate), Msc(2-Methylsulfoethylcarbamate) Nsc(4-nitrophenylethylsulfonyl-ethyloxycarbonyl]. Preferred PG1 and X1 protecting groups are selected from “Protective Groups in Organic Synthesis,” Green and Wuts, Third Edition, Wiley-Interscience, (1999) with the most preferred being Fmoc and Nsc. Examples of preferred protecting groups for PG2 include, but are not limited to [Acm(acetamidomethyl), MeOBzl or Mob(p-Methoxybenzyl), MeBzl(p-Methylbenzyl), Trt(Trityl),Xan(Xanthenyl),tButhio(s-t-butyl),Mmt(p-Methoxytrityl),2 or 4 Picolyl(2 or 4 pyridyl)),Fm(9-Fluorenylmethyl), tBu(t-Butyl),Tacam(Trimethylacetamidomethyl)] Preferred protecting groups PG2 and X2 are selected from “Protective Groups in Organic Synthesis,” Green and Wuts, Third Edition, Wiley-Interscience, (1999), with the most preferred being Acm, Mob, MeBzl, Picolyl.

Orthogonal protection schemes involves two or more classes or groups that are removed by differing chemical mechanisms, and therefore can be removed in any order and in the presence of the other classes. Orthogonal schemes offer the possibility of substantially milder overall conditions, because selectivity can be attained on the basis of differences in chemistry rather than reaction rates.

TABLE IV

| | |
|---|---|
| 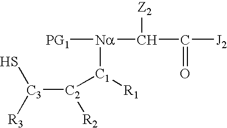 | 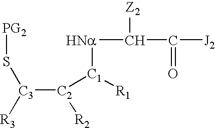 |

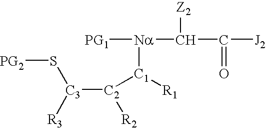

The protected forms of the Nα-substituted 2 or 3 chain alkyl or aryl thiols of the invention can be prepared as in Schemes I and II above.

The compounds of the present invention may be produced by any of a variety of means, including halogen-mediated amino alkylation, reductive amination, and by the preparation of Nα-protected, N-alkylated, S-protected, amino alkyl- or aryl-thiol amino acid precursors compatible with solid phase or solution amino acid or peptide synthesis methods. When desirable, resolution of the racemates or diastereisomers produced to give compounds of acceptable chiral purity can be carried out by standard methods.

As noted above, Nα-substituted 2 or 3 carbon chain alkyl or aryl thiols of acceptable chiral purity are preferred in some instances. As shown in Example 21, and in FIG. 5B, use of the Nα-1-(4-methoxyphenyl)-2-mercaptoethyl auxiliary in the preparation of cytochrome b562 yielded two ligation products (diastereoisomers) with overlapping purification profiles. Although removal of the Nα-auxiliary yields a single major product, a small percentage of deletion and side-reactant products will be present in the final product, which may be undesirable. For instance, the reductive amination synthetic route as described in Examples 4 through 6 employed for synthesis the Nα-1-(4-methoxyphenyl)-2-mercaptoethyl auxiliary employed in the cyt b562 synthesis inherently results in the production of both epimers at the chiral center C1. As noted above, when desirable, resolution of the racemates or diastereisomers produced to give compounds of acceptable chiral purity can be carried out by standard methods.

Standard approaches for obtaining Nα-auxiliaries of the invention of acceptable chiral purity are: (1) chiral chromatography; (2) chiral synthesis; (3) use of a covalent diasteriomeric conjugate; and (4) crystallization or other traditional separation methods to give enantiomerically pure chiral auxiliary. (See, e.g., Ahuja, Satinder. ‘Chiral separations. An overview.’ ACS Symp. Ser. (1991), 471(Chiral Sep. Liq. Chromatogr.), 1-26; Collet, Andre. “Separation and Purification of Enantiomers by Crystallization Methods”, In: Enantiomer (1999) 4:157-172; Lopata et al., J. Chem. Res. Minipprint (1984) 10:2930-2962; Lopata et al., J. Chem. Res. (1984) 10:2953-2973; Ahuja, Satinder. ‘Chiral separations and technology: an overview.’ Chiral Sep. (1997), 1-7; Chiral Separations: Applications and Technology. Ahuja, Satinder; Editor. USA. (1997), 349 pp. Publisher (ACS, Washington, D.C.)). All of these standard methods approaches can be used for resolution of racemates or diastereisomers to give compounds of acceptable chiral purity. For instance, crystallization can be employed for optical resolution of enantiomers. For chiral chromatography, it is well known that racemic mixtures can be separated into chirally pure enantiomers by means of preparative chromatography using chiral media. Thus, a racemic mixture produced by the reductive amination route for the total synthesis of chiral Nα-auxiliaries can be used to prepare each enantiomer in chirally pure form, for example, as illustrated below for an amino acid auxiliary (e.g., where R is amino acid side chain):

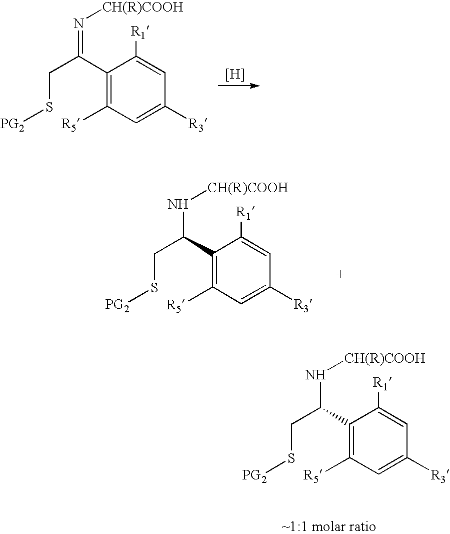

Either enantiomer may be obtained in chirally pure form, or both may be obtained in chirally pure form. Either enantiomer may be used to form chirally-pure auxiliary modified components, such as peptide segments (i.e. two chirally pure epimers), which can be rigorously purified without interference from the presence of the other epimer and its impurities. Note that unless some provision is made for using both enantiomers, 50% of the total mass of the auxiliary will be wasted. For example, the two chirally pure auxiliary-modified peptide segments can then used in separate ENCL reactions, to give chirally pure auxiliary-modified ligation product mixtures. After separate purifications, the auxiliary group is removed from the epimer ligation products (either separately or after being combined) to give the same native structure, ligated product, which is then subjected to purification.

For chiral synthesis, a preferred method employs an enantiomerically pure, chiral starting material, as illustrated below for a para-methoxyphenyl substituted Nα-2 carbon auxiliary: 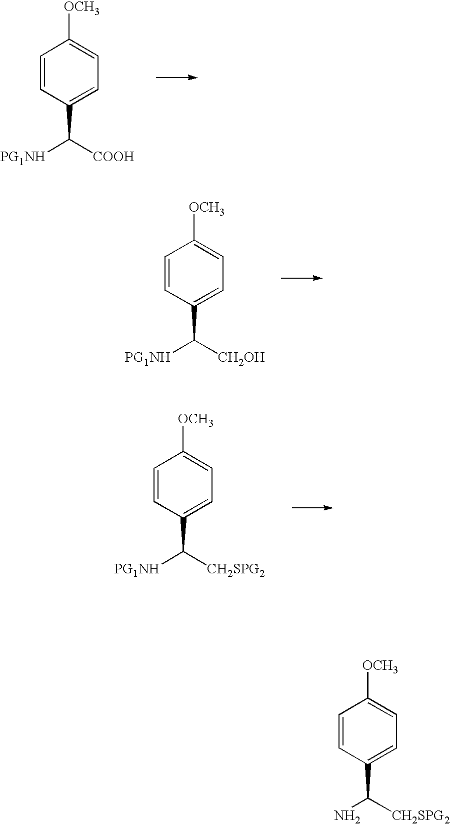 [PG₁=Boc or Fmoc; PG₂=(4Me)Benzyl or (4MeO)Benzyl]

The resulting chirally pure precursor compound can then be used to make either a protected (N-substituted) amino acid, viz.:

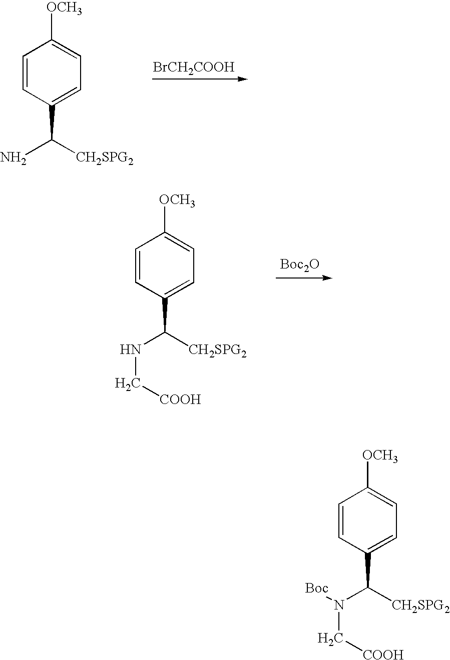

or used directly in the ‘sub-monomer’ peptoid route, viz:

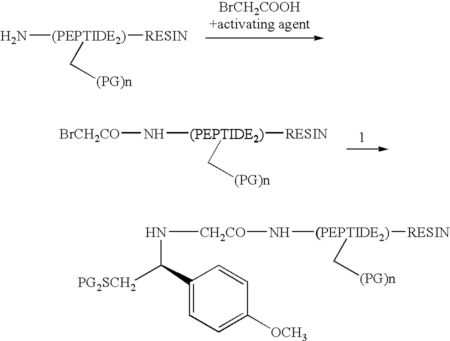

to form the auxiliary-modified peptide on a polymer support. Subsequent deprotection/cleavage gives the auxiliary-modified peptide segment in chirally-pure form, viz.:

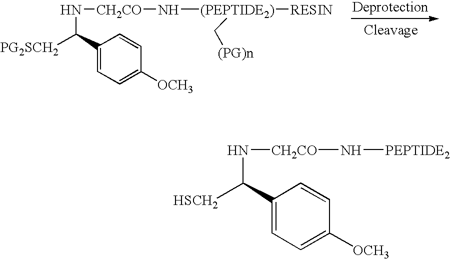

Thus chirally pure Nα-auxiliaries of the invention can be made from the readily available para-substituted phenylgylcine(s) of known chirality, thus predetermining the chirality and chiral purity of the resulting auxiliary.

Alternatively, another preferred embodiment employs enantioselective synthesis employing asymmetric reduction to yield the auxiliary, for example as illustrated below:

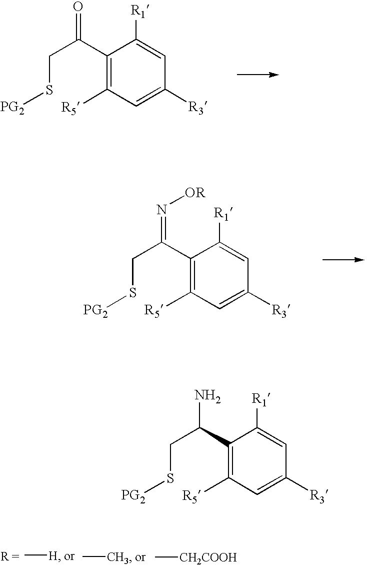

(or, opposite enantiomer)

Asymmetric reduction can also be used for enantioselective synthesis to yield an Nα-auxiliary-modified amino acid, such as for glycine illustrated below, viz.:

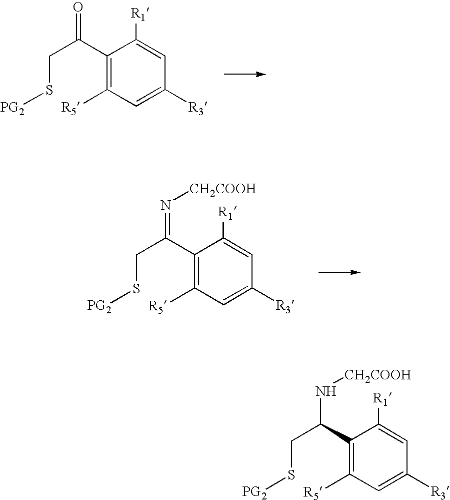

(or, other enantiomer)

While a suitably executed asymmetric synthesis will give a considerable excess of one enantiomer over the other, nonetheless H is expected that there will be present amounts of the other enantiomer. This can be addressed using a chiral purification step in order to obtain the majority enantiomer in pure form. The main benefits of an enantioselective synthetic route are that the chiral chromatographic separation is easier, and that large amounts of material are not discarded (wasted).

Another preferred standard technique is resolution by use of a covalent diasteriomeric conjugate. In general, this approach employs a chiral amino acid (e.g. Ala) to modify a racemic auxiliary mixture, and separation of the resulting diastereomers by standard (non-chiral) chromatography methods, such as illustrated below. For instance, the racemic auxiliary 1 can be converted to a mixture of diastereomers by covalent incorporation of a second chiral center

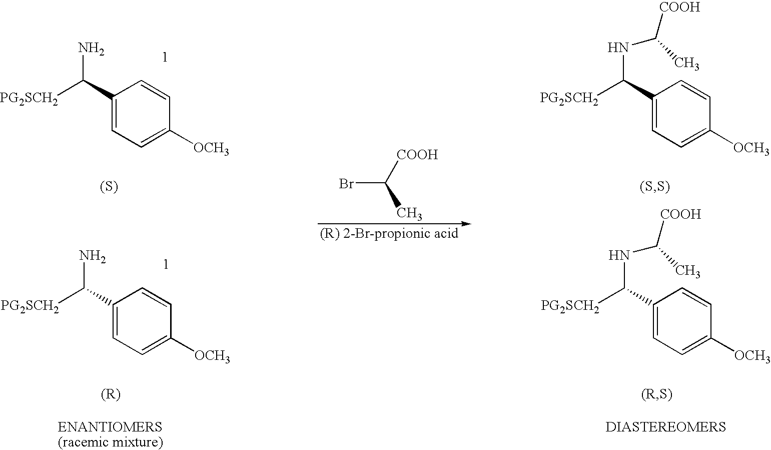

In this case, the racemic mixture is reacted, by means of an SN2 nucleophilic reaction (with inversion), with (R)2-Br-propionic acid, to yield the pair of diastereomers shown. In effect, we have generated L-Ala with an N-linked chiral thiol-containing auxiliary.

As diastereomers, these two compounds will typically exhibit different chromatographic behavior under achiral chromatography conditions, and thus be separable under practical preparative conditions to give the pure, distinct epimers. After suitable protection of the N^{α} moiety, each compound can be used to generate an unprotected or partially protected chirally pure auxiliary-modified peptide segment with an N-terminal Ala residue.

The protected Nα-substituted components of the invention are particularly useful for rapid automated synthesis using conventional peptide synthesis and other organic synthesis strategies. They also expand the utility of chemical ligation to multi-component ligation schemes, such as when producing a polypeptide involving orthogonal ligation strategies, such as a three or more segment ligation scheme or convergent ligation synthesis schemes.

For instance, the extended native chemical ligation method and compositions of the invention can be employed in conjunction with nucleophile stable thioester generating methods and thioester safety-catch approaches, such as the orthothioloester and carboxyester thiols described in co-pending application PCT application Serial No. [Not yet assigned] filed Aug. 31, 2001, and U.S. provisional patent application Serial No. 60/229,295 filed Sep. 1, 2000, which are incorporated herein by reference. Briefly, the nucleophile-stable thioester generating compounds comprise an orthothioloester or a carboxyester thiol; these compounds have wide applicability in organic synthesis, including the generation of peptide-, polypeptide- and other polymer-thioesters. The nucleophile-stable thioester generating compounds are particularly useful for generating activated thioesters from precursors that are made under conditions in which strong nucleophiles are employed, such as peptides or polypeptides made using Fmoc SPPS, as well as multi-step ligation or conjugation schemes that require (or benefit from the use of) compatible selective-protection approaches for directing a specific ligation or conjugation reaction of interest. The nucleophile-stable orthothioloesters have the formula X—C(OR′)₂—S—R, where X is a target molecule of interest optionally comprising one or more nucleophile cleavable protecting groups, R′ is a nucleophile-stable protecting group that is removable under non-nucleophilic cleavage conditions, and R is any group compatible with the orthothioloester —C(OR′)—S—. Nucleophile-stable orthothioloester thioester-generating resins also are provided, and have the formula X—C(OR′)₂—S—R-linker-resin or X—C((OR₁′-linker-resin )(OR₂′))—SR, where X, R′ and R are as above, and where the linker and resin are any nucleophile-stable linker and resin suitable for use in solid phase organic synthesis, including safety-catch linkers that can be subsequently converted to nucleophile-labile linkers for cleavage. The nucleophile-stable orthothioloesters can be converted to the active thioester by a variety of non-nucleophilic conditions, such as acid hydrolysis conditions. The nucleophile-stable carboxyester thiols have the formula X—C(O)—O—CH(R″)—CH₂)ₙ—S—R′″, where X is a target molecule of interest comprising one or more nucleophile-labile protecting groups, R″ is a non-nucleophile stable group, n is 1 or 2, with n=1 preferred, and R′″ is hydrogen, a protecting group or an acid- or reduction-labile or safety catch linker attached to a resin or protecting group that is removable under non-nucleophilic conditions. Nucleophile-stable carboxyester thiol-based thioester-generating resins also are provided, and have the formula X—C(O)—O—CH(R″)—CH2n-S-linker-resin or X—C(O)—O—CH(R″-linker-resin)-CH2n—SR′″, where X, R″, n and R′″ are as above, and where the linker and resin are any nucleophile-stable linker and resin suitable for use in solid phase organic synthesis. The nucleophile-stable carboxyester thiols can be converted to the active thioester by addition of a thiol catalyst, such as thiophenol. Thus the extended native chemical ligation methods and compositions of the present invention can be employed in multi-segment convergent ligation techniques, where a one end of a target compound can bear a protected or unprotected Nα-2 or 3 carbon chain alkyl or aryl thiol of the present invention, and the other end a orthothioloester or carboxyester thiol moiety for subsequent conversion to the active thioester and ligation.

It will also be appreciated that the Nα-2 or 3 carbon chain alkyl or aryl thiol of the present invention can be employed in combination with other ligation methods, for example, such as native chemical ligation (Dawson, et al., Science (1994) 266:776-779; Kent, et al., WO 96/34878), extended general chemical ligation (Kent, et al., WO 98/28434), oxime-forming chemical ligation (Rose, et al., J. Amer. Chem. Soc. (1994) 116:30-33), thioester forming ligation (Schnölzer, et al., Science (1992) 256:221-225), thioether forming ligation (Englebretsen, et al., Tet Letts. (1995) 36(48):8871-8874), hydrazone forming ligation (Gaertner, et al., Bioconj. Chem. (1994) 5(4):333-338), and thiazolidine forming ligation and oxazolidine forming ligation (Zhang, et al., Proc. Natl. Acad. Sci. (1998) 95(16):9184-9189; Tam, et al., WO 95/00846) or by other methods (Yan, L. Z. and Dawson, P. E., “Synthesis of Peptides and Proteins without Cysteine Residues by Native Chemical Ligation Combined with Desulfurization,” J. Am. Chem. Soc. 2001, 123, 526-533, herein incorporated by reference; Gieselnan et al., Org. Lett. 2001 3(9):1331-1334; Saxon, E. et al., “Traceless” Staudinger Ligation for the Chemoselective Synthesis of Amide Bonds. Org. Left. 2000, 2, 2141-2143). Also contemplated by the present invention is the substitution of selenium in place of the thiol sulfur in the Nα-2 or 3 carbon chain alkyl or aryl thiol of the invention.

The methods and compositions of the invention have many uses. The methods and compositions of the invention are particularly useful for ligating peptides, polypeptides and other polymers. The ability to carry out native chemical ligation at practically any amino acid, including the naturally occurring as well as unnatural amino acids and derivatives expands the scope of native chemical ligation to targets that are missing suitable cysteine ligation sites. The invention also can be used to ligate polymers in addition to peptide or polypeptide segments when it is desirable to join such moieties through a linker having an Nα-substituted or totally native amide bond at the ligation site. The invention also finds use in the production of a wide range of peptide labels for expressed-protein ligation (EPL) applications. For instance, EPL-generated thioester polypeptides can be ligated to a wide range of peptides via an Nα-substituted alkyl or aryl thiol amide bond or a completely native amide bond, depending on the intended end use. The invention also can be exploited to produce a variety of cyclic peptides and polypeptides, having a native amide bond at the point of cyclization even for peptides and polypeptides that do not contain cysteine. For instance, this is significant as most cyclic peptides, such as antibiotics and other drugs generated by industry standards do not contain a cysteine residue that can be used to form a native amide bond at the cyclizing (i.e., head-to-tail) ligation site.

All publications and patent applications mentioned in this specification are herein incorporated by reference to the same extent as if each individual publication or patent application was specifically and individually indicated to be incorporated by reference.

### EXAMPLES

The following preparations and examples are given to enable those skilled in the art to more clearly understand and to practice the present invention. They should not be considered as limiting the scope of the invention, but merely as being illustrative and representative thereof.

Abbreviations

| | |
|---|---|
| Acm | acetamidomethyl |
| Aloc | allyoxycarbonyl |
| BOP | benzotriazol-1-yloxytris (dimethylamino) phosphonium |
| | hexafluorophosphate |
| Br, Cl Z | Br, Cl Benzylcarbamate |
| DCM | dichloromethane |
| DDE | 4,4-dimethyl-2,6-dioxocycloex 1-ylidene |
| DIPCDI | N,N-diisopropylcarbodiimide |
| DIPEA | N,N-diisopropylethylamine |
| DMAP | 4-dimethylaminopyridine |
| DMF | N,N-dimethylformamide |
| DMSO | dimethylsulfoxide |
| EtOH | ethanol |
| Fmoc | 9-fluorenylmethoxycarbonyl |
| FM | 9-Fluorenylmethyl |
| HATU | (N-[(dimethylamino)-1H-1,2,3-triazol [4,5-b] |
| | pyridiylmethylene]-N-methylmethanaminium |
| | hexafluorophosphate N-oxide). |
| HBTU | N-[(1-H-benzotriazol-1-yl)(dimethylamine) methylene]-N- |
| | methylmethanaminium hexafluorophosphate N-oxide |
| | previously named 0-(benzotriazol-1-yl)-1,1,3,3- |
| | tetramethyluronium hexafluorophosphate |
| HF | hydrofluoric acid |
| HMP resin | 4-hydroxymethylphenoxy resin; palkoxybenzyl alcohol |
| | resin; or Wang resin |
| HOAt | 1-hydroxy-7-azabenzotriazole |
| HOBt | 1-hydroxybenzotriazole |
| Mbh | dimethoxybenzhydryl |
| MBHA resin | 4-methylbenzhydrylamine resin |
| Meb | p-MethylBenzyl |
| MMA | N-methylmercaptoacetamide |
| Mmt | p-Methoxytriityl |
| Mob | p-MethoxyBenzyl |
| Msc | 2-Methylsulfoethylcarbamate |
| Msz | 4-Methylsulfinylbenzylcarbamate |
| Mtr | 4-methoxy-2,3,6-trimethylbenzene sulfonyl |
| NMM | Nmethylmorpholine |
| NMP | N-methylprrolidone, N-methyl-2-pyrrolidone |
| Nsc | 4-nitrophenylethylsulfonyl-ethyloxycarbonyl |
| OPfp | pentafluorophenyl ester |
| OtBu | tert-butyl ester |
| PAC | peptide acid linker |
| PAL | peptide amide linker |
| Pbf | 2,2,4,6,7-pentamethyldihydrobenzofuran-5-sulfonyl |
| PEG-PS | polyethylene glycol-polystyrene |
| Picolyl | methyl-pyridyl |
| Pmc | 2,2,4,6,8-pentamethylchroman-6-sulfonyl |
| PyAOP | 7-azabenzotroazol-1-1yloxtris (pyrrolidino) phosphonium |
| | hexafluorophosphate |
| S-tBu | tert-butyl-thio |
| Tacam | Trimethylacetamidomethyl |
| tBoc | tert-butyloxycarbonyl |
| TBTU | O-(benzotriazol-1-yl)-1,1,3,3-tetramethyl uronium |
| | tetrafluoroborate |
| tBu | tert-butyl |
| TFA | trifluoroacetic acid |
| Tis | Trisisopropylsilane |
| Tmob | 2,4,6-trimehoxybenzyl |
| TMOF | trimethylorthoformate |
| Troc | 2,2,2Trichloroethylcarbamate |
| Trt | triphenylmethyl |

### Example 1

### General Materials and Methods

Peptides were synthesized in stepwise fashion on a modified ABI 430A peptide synthesizer by SPPS using in situ neutralization/HBTU activation protocols for Boc-chemistry on PAM resin or thioester-generating resin following standard protocols (Hackeng et al., supra; Schnolzer et al., (1992) Int. J. Pept. Prot. Res., 40:180-193; and Kent, S. B. H. (1988) Ann. Rev. Biochem. 57, 957-984). After chain assembly the peptides were deprotected and simultaneously cleaved by treatment with anhydrous hydrogen fluoride (HF) with 5% p-cresol and lyophilized and purified by preparative HPLC. Boc protected amino acids were obtained from Peptides International and Midwest Biotech. Trifluoroacetic add (TFA) was obtained by Halocarbon. Other chemicals were from Fluka or Aldrich. Analytical and preparative HPLC were performed on a Rainin HPLC system with 214 nm UV detection using Vydac C4 analytical or preparative. Peptide and protein mass spectrometry was performed on a Sciex API-I electrospray mass spectrometer.

### Example 2

### Preparation of 2(4′methoxybenzylthio)benzylbromide

2-Hydroxymethylthiophenol, 10 mmol 1.4 g, was reacted in 10 ml of DMF with 10 mmol of 4-methoxybenzylchloride and 1.75 ml of DIEA at room temperature. The reaction is completed in 10 minutes 0.50 ml of water at pH 3 were added. The product was extracted with ethyl acetate and dried over sodium sulfate. The obtained crude oil was then reacted with 11 mmol of carbon tetrabromide(3.64 g) and 11 mmol of triphenylphosphine(2.88 g) in 20 ml of THF. After overnight reaction the THF was evaporated. The product was purified with silica gel chromatography using hexanes/ethyl acetate 6/1 as mobile phase. Recovered 1.8 g.

### Example 3

### Preparation of Nα(2-mercaptobenzyl) glycine-peptide

To a resin bound peptide with N-terminal Boc-protected Ala(78 mg), neat TFA was added to remove the Boc group. Using standard chemistry protocols BocGlycineOSuccinimide was coupled to the resin. After the coupling was completed Boc group was removed and the resin was neutalized with 2 washes with 10% Diisopropylethylamine in DMF.The resin was then washed with DMF and DMSO. Then 9 mg of 2(4′methoxybenzylthio)benzylbromide in 0.2 ml of DMSO and 0.01 ml of Diisopropylethylamine were added. The mixture reacted for 12 hrs at room temperature. The peptide was cleaved and deprotected in HF conditions using standard protocols. The peak with correct mass of 2,079 Da was about 12% (measured by HPLC) of all peptidic material. The correct peptide was purified using standard semi-preparative HPLC.

### Example 4

### Preparation of 4′-Methoxy 2(4′methylbenzylthio) acetophenone

4-methylbenzylmercaptan, 4 mmol, 0.542 ml and 4′methoxy 2bromoacetophenone 4 mmol, 916.3 mg were dissolved in 4 ml DMF. Then diisopropylethylamine, 4 mmol 0.7 ml was added. The mixture was stirred at room temperature for one hour. The mixture was poured in diluted HCl and extracted with ethylacetate and dried over sodium sulfate. The oil was dissolved in ethylacetate and precipitated by addition of petroleum ether, with recovery of 450 mg of a white solid.

### Example 5

### Preparation of 1 amino,1(4-methoxyphenyl),2(4-methylbenzylthio) ethane

4′-Methoxy 2(4′methylbenzylthio) acetophenone 1.44 mmol, 411 mg and aminoxyacetic acid 4.3 mmol, 941 mg were dissolved in 20 ml of TMOF and 0.047 ml of methanesulfonic acid was added as catalyst at room temperature. After 48 hours, the solvent was evaporated and the residue taken up in ethylacetate, washed with 1M monohydrogenpotassium sulfate and dried over sodium sulfate. The crude product was purified with silica gel chromatography, and 200 mg of oxime complex obtained. T200 mg of this oxime complex, 0.556 mmol was dissolved in 2 ml of THF, followed by the addition of 1.67 ml of 1M BH3/THF complex. After 27 hours no starting material was left. 3 ml of water were added and 1.5 ml of 10N sodium hydroxide was added. The mixture was refluxed for 1 hour. The mixture was then extracted with ethylacetate (4×) and dried over sodium sulfate. The final product (40 mg) was then purified using silica gel chromatography.

### Example 6

### Preparation of Nα 1-(4Methoxyphenyl) 2-mercapto ethane glycine-peptide

To a resin bound model peptide with an N-terminal Boc protected Ala (78 mg), neat TFA was added to remove Boc group. Using standard chemistry protocols bromoacetic acid was coupled to the resin. Then 1 amino,1(4-methoxyphenyl),2(4-methylbenzylthio) ethane 17 mg in 0.3 ml of DMSO with 0.010 ml of diisopropylethylamine were added to the resin. After overnight reaction the resin was washed and the peptide was cleaved and deprotected using standard HF protocol. The desired product was then purified using semi-preparative HPLC.

### Preparation of Another Nα 1-(4-Methoxyphenyl) 2-mercapto ethane glycine-peptide

To a model peptide resin of sequence S—Y—R—F-L-Polymer 0.1 mmol, bromo acetic acid was coupled using standard coupling protocol. After coupling the resin was washed with DMSO. Then 1 amino,1(4-methoxyphenyl),2(4-methylbenzylthio) ethane 32.5 mg, 0.12 mmol in 0.3 ml DMSO and 0.025 ml of diisopropylethylamine were added. The mixture was kept reacting overnight. The peptide was cleaved and deprotected using standard HF procedure. The HPLC of the crude cleavage showed the desired product (MW 2,122) in 60% of the total product. The n-alkylethanethio group was found to be 97% stable in HF.

### Example 7

### Preparation of 2′,4′-Dimethoxy 2(4′methylbenzylthio) acetophenone

4-methylbenzylmercaptan, 3.94 mmol, 0.534 ml and 2′,4′ dimethoxy 2-bromoacetophenone 3.86 mmol, 1 g were dissolved in 4 ml DMF. Then Diisopropylethylamine, 3.94 mmol 0.688 ml was added. The mixture is stirred at room temperature for 24 hrs. The mixture is poured in 1M solution of potassium monohydrogensulfate and extracted with ethylacetate and dried over sodium sulfate. After evaporation, the residual oil was dissolved in ethylacetate and precipitated by addition of petroleum ether, which yielded 616 mg of a white solid.

### Example 8

### Preparation of 1 amino,1(2,4-dimethoxyphenyl),2(4-methylbenzylthio) ethane

2′,4′-Dimethoxy 2(4′methylbenzylthio) acetophenone 0.526 mmol, 166 mg and aminoxyacetic acid 1.59 mmol, 345 mg were dissolved in 6 ml of TMOF and 0.034 ml of methanesulfonic acid was added as catalyst at room temperature. After 31 hrs. the solvent was evaporated and taken up in ethylacetate, washed with 1M monohydrogenpotassium sulfate and dried over sodium sulfate. The crude product was then purified with silica gel chromathography, yielding 126 mg (61% yield) of oxime complex. The 126 mg of oxime complex, 0.324 mmol was dissolved in 1.5 ml of THF. Then 0.973 ml of 1M BH3/THF complex was added.

After 54 hrs starting material was still found and 0.5 ml of 1M BH3/THF complex was added. After 3 more days, total of 6 days reaction 3 ml of water were added and 1 ml of 10N sodium Hydroxide was added. The mixture was refluxed for 1 h. The mixture was then extracted with ethylacetate (4×) and dried over sodium sulfate. The final product (43 mg) was then purified using silica gel chromatography.

### Example 9

### Preparation of Nα 1-(2,4-Dimethoxyphenyl) 2-mercapto ethane glycine-peptide

To a resin bound peptide of the sequence S—Y—R—F-L-Polymer, 0.1 mmol, bromo acetic acid was coupled using a standard coupling protocol. After coupling the resin was washed with DMSO. Then 1 amino,1(2′,4′-dimethoxyphenyl),2(4-methylbenzylthio) ethane 36 mg, 0.12 mmol in 0.3 ml DMSO and 0.025 ml of diisopropylethylamine were added. The mixture was kept reacting overnight. The peptide was cleaved and deprotected using standard HF procedure. The HPLC of the crude cleavage showed the desired product (MW 938) in 42% of the total product. The n-alkylethanethiol group was found to be 92% stable in HF.

### Example 10

### Ala-Gly Chemical Ligation of C-terminal SDF1-alanine-thioester and N-terminal Nα (2-mercaptobenzyl) glycine-peptide

For 6-member rearrangement ligation, 1 mg of C-terminal Ala thioester fragment (MW 4429) of SDF1-α, and 0.6 mg of N-terminal Nα (2-mercaptobenzyl) glycine fragment (MW 2079) of SDF1-α, were dissolved in 100 μl of 6 M guanidinium buffer pH 7.0 and 1 μl of thiophenol was added. After 2 days at room temperature (˜25° C.), formation of the desired ligation product (MW 5472) was confirmed by ES-MS. The reaction mixture was then incubated at 40° C. for an additional 24 hours, and the yield of desired ligation product determined based on the ratio between product and the non-reacted C-terminal fragment as measured by HPLC integration. The observed yield was about 40%.

### Example 11

### Ala-Gly Chemical Ligation of C-terminal SDF1-alanine-thioester and N-terminal Nα 1-(4-methoxyphenyl) 2-mercaptoethane glycine-peptide

For 5-member rearrangement ligation, 1 mg of C-terminal alanine-thioester fragment (MW 4429) of SDF1, and 1 mg of an N-terminal peptide Model (MW 2122) having an N-terminal glycine comprising an Nα 1-(4-methoxyphenyl) 2-mercaptoethane group, were dissolved in 100 μl of 6 M guanidinium buffer pH 7.0 [and 1 μl of thiophenol]. The reaction mixture was incubated at room temperature (˜25° C.), and the ligation reaction monitored. After 8 hours, formation of the desired ligation product (MW 5515) was confirmed by ES-MS. After 3 days, yield of the desired ligation product was about 45% based on the ratio between product and the non-reacted N-terminal fragment. After 3 days at room temperature, followed by further incubation at 40° C. for an additional 24 hours, the yield was 65%. After 3 days at room temperature, followed by further incubation at 40° C. for an additional 48 hours, the yield increased to about 70%.

For 5-member rearrangement ligation, 0.5 mg of C-terminal Ala thioester fragment (MW 4429) of SDF1-α, and 0.5 mg of an N-terminal fragment (MW 2122) having an N-terminal glycine comprising an Nα 1-(4-methoxyphenyl) 2-mercaptoethane group, were dissolved in 100 μl of 6 M guanidinium buffer pH 8.2 and 1 μl of thiophenol. The reaction mixture was then incubated at room temperature (˜25° C.), followed by the addition of another 1 μl of thiophenol after 6 hours. After 24 hours, the yield of desired product was about 60%.

### Example 12

### Gly-Gly Chemical Ligation of C-terminal glycine-thioester and N-terminal Nα-(2-mercaptobenzyl) glycine-peptide

For 6-member rearrangement ligation, 3.5 mg of C-terminal Gly thioester fragment (MW 1357) of a decamermodel peptide, and 2 mg of N-terminal Nα (2-mercaptobenzyl) glycine fragment (MW 2079) of a model peptide with 3 HisDnp, were dissolved in 200 μl of 6 M guanidinium buffer pH 7.9 and 2 μl of thiophenol was added. The mixture was incubated at 33° C. for 60 hours. Formation of the desired ligation product (MW 2631) was confirmed by ES-MS, with an observed yield of about 40% based on the ratio between product and the non-reacted N-terminal fragment.

### Chemical Ligation of C-terminal glycine-thioester peptide and Nα 1-(4-methoxyphenyl) 2-mercaptoethane glycine-peptide

For 5-member rearrangement ligation, 2 mg of C-terminal Gly thioester fragment (MW 1357) and 2.5 mg of an N-terminal fragment (MW 2122) of a model peptide with 3 HisDnp comprising an Nα 1-(4methoxyphenyl) 2-mercaptoethane group, were dissolved in 100 μl of 6 M guanidinium buffer pH 7.0 with 1 μl of thiophenol. The reaction mixture was incubated at room temperature (˜25° C.), and the ligation reaction monitored. Formation of the desired ligation product (MW 2675.9) was confirmed by ES-MS after 3 and 8 hours of incubation. After 24 hours, yield of the desired ligation product was about 40% based on the ratio between product and the non-reacted N-terminal fragment. The pH was then raised to 8.2 by addition of solid sodium bicarbonate, and the reaction mixture incubated for an additional 24 hours, resulting in a yield of 88%.

### Example 13

### Ala-Gly Chemical Ligation of Larc-alanine-thioester with Nα 1-,(4-methoxyphenyl) 2-mercapto ethane glycine-peptide

A mouse Larc 1-31 Ala C terminal peptide thioester 3 mg (MW 3609) and model peptide Nα 1-,(4-methoxyphenyl) 2-mercapto ethane glycine-S—Y—R—F-L 1 mg (MW 908) were dissolved in 0.15 ml 6 molar guanidinium buffer pH8.2 and 0.03 ml thiophenol. After overnight stirring the ligation was 81% complete and after 40 hrs 92% complete based on consumption of peptide thioester. Expected ligated product 4312 Da, found 4312 Da.

### Example 14

### Ala-Gly Chemical Ligation of Larc 1-31-alanine-thioester with Nα 1-(2,4-Dimethoxyphenyl) 2-mercapto ethane glycine-peptide

Mouse Larc 1-31 Ala C terminal peptide thioester 3 mg (MW 3609) and model peptide Nα 1-(2,4-dimethoxyphenyl) 2-mercapto ethane glycine-S—Y—R—F-L 1 mg (MW 938) were dissolved in 0.15 ml 6 molar guanidinium buffer pH8.2 and 0.03 ml thiophenol. After overnight stirring the ligation was 73% complete, and after 40 hrs 85% complete based on consumption of peptide thioester. The calculated and experimental masses of the ligation product were both 4342 Da.

### Example 15

### Gly-Gly Chemical Ligation of C-terminal tripeptide glycine-thioester and N-terminal Nα-1-(2,4-dimethoxyphenyl) 2-mercapto ethane glycine-peptide

Peptide fragment FGG-thioester 0.8 mg and model peptide Nα 1-(2,4-dimethoxyphenyl) 2-mercapto ethane glycine-S—Y—R—F-L 1 mg (MW 938) were dissolved in 0.1 ml of 6M guanidinium buffer pH 8.2 and 0.02 ml of thiophenol. After overnight stirring the reaction was completed quantitatively. The calculated and experimental masses of the ligation product were 1199.4 Da and 1195.5 Da, respectively.

### Example 16

### Removal of 1-(2,4-Dimethoxyphenyl) 2-mercapto ethane from Ligation Product

1 mg of purified ligation product from Example 15 was dissolved in 0.95 ml of TFA and 0.025 ml of water and 0.025 ml of TIS. After 1 h. the solvent was evaporated and a 50% solution water/acetonitrile was added and the mixture lyophilized. The cleavage is greater than 95% complete by HPLC. The calculated and experimental mass was 1003 Da.

### Example 17

### Gly-Gly Chemical Ligation of C-terminal tripeptide glycine-thioester and N-terminal Nα 1-,(4-methoxyphenyl) 2-mercapto ethane glycine-peptide

A tripeptide peptide fragment thioester, FGG-thioester 1.6 mg and model peptide Nα 1-(4-methoxyphenyl) 2-mercapto ethane glycine-S—Y—R—F-L 2 mg (MW 908) were dissolved in 0.2 ml of 6M guanidinium buffer pH 8.2 and 0.04 ml of thiophenol was added. After overnight stirring the reaction was completed quantitatively. Expected MW for ligated product 1169.4 Da, found 1169.5 Da

### Example 18

### Removal of the 1-(,4-methoxyphenyl) 2-mercapto ethane Group after Ligation

Purified ligation product from Example 17 was treated with HF 5% p cresol at −2° C. for 1 hour. After HF evaporation, the ligation product was precipitated with ether. The crude peptide was taken up in 50% water/acetonitrile 0.1% TFA and injected on HPLC. The major peak >80% showed the expected molecular weight for the cleaved peptide (expected mass 1003 Da, found 1003 Da).

### Example 19

### His-Gly Chemical Ligation of C-terminal histidine-thioester and N-terminal Nα 1-(2,4-Dimethoxyphenyl) 2-mercapto ethane glycine-peptide

Peptide fragment TBP-A 1-67 CαHis thioester 4 mg and model peptide Nα 1-(2,4Dimethoxyphenyl) 2-mercapto ethane Glycine-S—Y—R—F-L 1 mg (MW 938) were dissolved in 0.1 ml of 6M guanidinium buffer pH 8.2 and 0.02 ml of thiophenol. After overnight stirring the reaction was 87% complete based on the consumption of the peptide thioester. Expected molecular weight for the ligated product 9220 Da, and found 9220 Da.

### Example 20

### Removal of the 1-(2,4-Dimethoxyphenyl) 2-mercapto ethane Group After Ligation

Purified peptide fragment after H-G ligation 2 mg was dissolved in 0.95 ml of TFA and 0.025 ml of water and 0.025 ml of TIS. After 1 h. the solvent was evaporated and to the residue was added a 50% solution water/acetonitrile and the mixture was lyophilized. The cleavage is >90% complete by HPLC. Expected MW 9023 Da, found 9024 Da.

### Example 21

### Synthesis of Cytochrome b562 by Extended Native Chemical Ligation

3 mg of Cytochrome 1-63 C-terminal thioester MW 7349 0.4 μmol and 1.5 mg of N-terminal Nα 1-,(4methoxyphenyl) 2-mercapto ethane glycine Cytochrome b562 residues 64-106 MW 4970 0.3 μmol were dissolved in 0.1 ml of 6M guanidinium ligation buffer pH 7with 0.002 ml of thiophenol as catalyst. See FIG. 5A. After 24 hrs 0.025 ml of 2-mercapto ethanol were added to the mixture and kept reacting for 45 minutes, then 15 mg of TCEP were added and after additional 30 minutes stirring the ligation mixture was loaded onto a semi-preparative HPLC. After the break through was eluted and the mixture was then desalted all the components of the ligation mixture were eluted by ramping the gradient to 65% B and collected in a sole vial. The analytical HPLC of the desalted material showed the ligation was greater than 90% complete based on the consumption of the C terminal peptide. The HPLC showed two major peaks (diastereoisomers) with calculated and expected mass of 11,946 Da. See FIGS. 5B and 6A.

The amino acid sequence for Cytochrome b562 (1-106) is shown below:

| | |
|---|---|
| ADLEDNMETL NDNLKVIEKA DNAAQVKDAL TKMRAAALDA | (SEQ ID NO:1) |
| QKATPPKLED KSPDSPEMKD FRHGFDILVG QIDDALKLAN | |
| EGKVKEAQAA AEQLKTTRNA YHQKYR | |

Calculated Mass (average isotope composition) 11780.3 Da

N-terminal Group: Hydrogen C-terminal Group: Free Acid MH+Monoisotopic Mass=11774.0088 amu·HPLC Index=249.80

MH+Average Mass=11781.2781 amu

Bull & Breese value=1.5360

Elemental Composition: C508 H830 N147 O168 S3

User-Defined Amino Acid Residues: B-HisDNP

### Example 22

### Removal of the 1-,(4-methoxyphenyl) 2-mercapto ethane Group from Ligated Cytochrome b562 Residues 1-106 and Generation of the Native Protein

The desalted solution was then lyophilized prior to removal of the 1-,(4-methoxyphenyl) 2-mercapto ethane group. Lyophilized material was treated with HF 95% 5% anisol and 1 mmol of cysteine (121 mg) for 1 h at −2° C. The HF was evaporated using standard protocols and then 100 ml of 50% Buffer B were added and the mixture was lyophilized. The mixture was then purified using semi-preparative HPLC giving 2 mg of purified single peak native Cytochrome 1-106 (56% of yield after purification), with calculated and experimental mass of 11,780 Da. See FIGS. 6B and 7A.

An analog of wild type cytochrome b562 was also synthesized in the same manner, and designated Slm7 cyt b562. The Slm7 cyt b562 mutant differed from the wild type by replacing methionine at position 7 with a selenomethionine (sulfur of methionine replaced with its lower cogener selenium). Circular dichroism was performed that indicated high α-helical content in both apo wild type b562 and apo Slm7 b562 (data not shown). ESMS also showed that both apo wild type b562 and apo Slm7 b562 had the expected molecular masses (data not shown). The apo proteins were reconstituted with heme (heme pH7 NaPi overnight, room temperature), and the resulting proteins purified with ion exchange FPLC (FPLC purification Resource Q, Tris HCL pH 8, NaCl gradient). For example, see FIG. 7B. UV-visible (optical) spectra of the heme-reconstituted proteins were found to be consistent with sulfur or selenium coordination to Fe (data not shown). A cyt b562 mutant with the non-coordinating isotere norleucine also is prepared in the same manner. Thus synthetic cytochromes were made using extended native chemical ligation, reconstituted with their heme active sites and fully characterized by biophysical methods. Accordingly, this example further demonstrates that peptides and proteins devoid of suitable cysteines for the original native chemical ligation approach can be made by extended native chemical ligation, and non-standard amino acids incorporated therein. For instance, the folding and reactivity of many cyt b562 mutants have been studied, but thus far unnatural axial ligands have remained unexplored. Together with extended native chemical ligation, the vast array of unnatural amino acids available should allow systematic tuning of the properties of these and other proteins.

### Example 23

### Lys-Gly Chemical Ligation of MCP 1-35-Lysine-thioester with Nα 1-,(4-methoxyphenyl) 2-mercapto ethane glycine-peptide

A MCP 1-35 Lys C terminal peptide thioester 3 mg and model peptide Nα 1-,(4-methoxyphenyl) 2-mercapto ethane glycine-S—Y—R—F-L 1 mg (MW 908) were dissolved in 0.15 ml 6 molar guanidinium buffer pH7 and adjusted to pH 7.2 by addition of Triethylamine and 0.03 ml thiophenol. After overnight stirring the ligation was 69% complete and after 40 hrs 76% complete based on consumption of peptide thioester. Expected ligated product 4893 Da, found 4893 Da

### Example 24

### Removal of the 1-(,4-methoxyphenyl) 2-mercapto ethane Group after Ligation)

Lyophilized de-salted crude from Example 23 (Lys-Gly ligation) was dissolved in 1 mg of TFA, 25 μl of Ethane di thiol, 50 μl of TIS. Then 150 μl of bromotrimethylsilane was added. The reaction was allowed to proceed for 2 hrs at room temperature (“rt”). The volatile components of the mixture were evaporated in vacuo, and the remaining oil was taken up in 6M Guanidinium buffer pH 7.5. The organic material was extracted with CHCl3. HPLC showed no more starting material, therefore the auxiliary group was successfully removed. The expected mass for the native sequence was 4726 Da, and a mass of 4725 Da was found.

### Example 25

### Comparison of Ligation Studies with GSYRFL Peptides

Comparison of 5-member rearrangement ligations studies with GSYRFL peptides from Examples 13-20 and 24 are summarized below in Table V.

TABLE V
Results of ligation studies with GSYRFL peptides

| | C-terminal | | | | |
|---|---|---|---|---|---|
| | Peptide | | | Ligation | Auxiliary |
| Model | (thioester | N-terminal | Reaction | Yield | Removal |
| Reaction | peptide) | Auxiliary | Time (h) | (%) | Conditions |
| 1 | Phe-Gly-Gly | I | 16 | >98% | HF |
| 2 | Phe-Gly-Gly | II | 16 | >98 | TFA |
| 3 | TBP-A 1-67 | II | 16 | 87 | TFA |
| | (His) | | | | |
| 4 | Mouse Larc | I | 16 | 81 | |
| | 1-31 (Ala) | | 40 | 92 | HF |
| 5 | Mouse Larc | II | 16 | 73 | |
| | 1-31 (Ala) | | 40 | 85 | TFA |
| 6 | MCP1 1-35 | I | 16 | 69 | |
| | (Lys) | | 40 | 76 | TFA/TMSBr |

Note:
N-terminal auxiliary I = Nα-1-(4-methoxyphenyl)-2-mercaptoethane glycine-SYRFL, and
N-terminal auxiliary II = Nα-1-(2,4-methoxyphenyl)-2-mercaptoethane glycine-SYRFL

### Example 26

### Preparation of BocGlycine N-1(4′-methoxyphenyl),2(4′-methylbenzylthio) ethane

4′-Methoxy 2(4′methylbenzylthio) acetophenone 2 mmol, 572 mg, and glycine ethyl ester HCl salt 2 mmol, 139.5 mg are suspended in 15 ml of DCM. DIEA 6 mmol, 1 g is added slowly and under nitrogen 1 ml of titanium tetrachloride (1 M solution) is added.

The reaction is kept for 2 days at room temperature. Then sodiumcyanoborohydride 6 mmol, 0.4 g in 2.5 ml of anhydrous methanol are added. The TLC shows approximately 40% of a spot of new product that after purification is identified by NMR as N-1(4-methoxyphenyl),2(4-methylbenzylthio) ethane Glycine ethyl ester.

1 mmol 374 mg of N-1(4-methoxyphenyl),2(4-methylbenzylthio) ethane Glycine ethyl ester is then dissolved in 2 ml of THF and 2 mmol of LiOH hydrate 83 mg are added to the solution. After overnight stirring the ester has been completely hydrolyzed. THF is removed in vacuo and the product is taken up in 2 ml of DMF, 5 mmol of ditbutyl dicarbonate 1.1 g are added and finally 3 mmol of DIEA, 0.45 ml. After overnight reaction diluted HCl water solution is added and the final product is extracted (3×) with ethyl acetate.

The invention now being fully described, it will be apparent to one of ordinary skill in the art that many changes and modifications can be made thereto without departing from the spirit or scope of the appended claims.

What is claimed is:

1. An N-substituted amide compound of the formula:
J1-C(O)—N(C1(R1)-C2-SH)-J2 I
or
J1-C(O)—N(C1(R1)-C2(R2)-C3(R3)-SH)-J2 II
where:
J1 and J2 are independently a peptide or polypeptide having one or more optionally protected amino acid side chains, or a moiety of such peptide or polypeptide, a polymer, a dye, a functionalized surface, a linker or detectable marker, or, any other chemical moiety compatible with chemical peptide synthesis or extended native chemical ligation; and
R1, R2 and R3 are independently H or an electron donating group conjugated to C1; with the proviso that at least one of said R1, R2 and R3 comprises said electron donating group conjugated to C1.

2. The N-substituted amide compound of claim 1, wherein said compound has said formula I.

3. The N-substituted amide compound of claim 2, wherein C1 (R1) is selected from the group consisting of A, B and C: 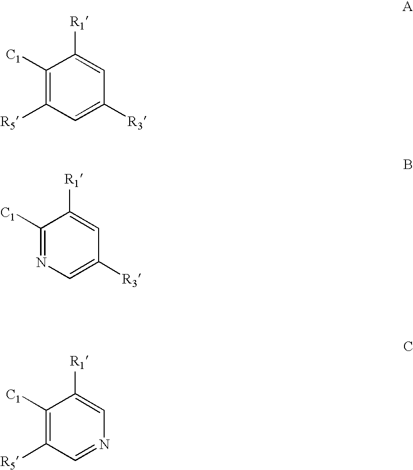
where R1′, R3′, and R5′ comprise electron-donating groups that may be the same or different.

4. The N-substituted amide compound of claim 1, wherein said compound has said formula II.

5. The N-substituted amide compound of claim 4, wherein C1(R1)-C2(R2)-C3(R3) is selected from the group consisting of D, E, F, G, H and I: 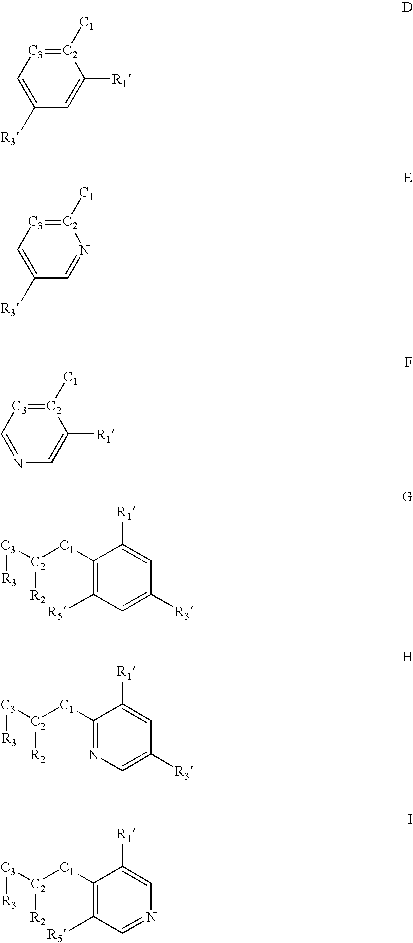
where one or more of R1′, R3′, and R5′ comprise an electron-donating group that may be the same or different.

6. The N-substituted amide compound of any of claims 1-5, wherein said substituted N is an Nα-substituted amide.

7. The N-substituted amide compound of any of claims 3 or 5, wherein at least one of R1′, R3′ and R5′ comprises a strong electron-donating group.

8. The N-substituted amide compound of claim 7, wherein said strong electron-donating group is selected from the group consisting of methoxy (—OCH₃), thiol (—SH), hydroxyl (—OH), and thiomethyl (—SCH₃).

9. The N-substituted amide compound of any of claims 3 or 5, wherein at least one of R1′, R3′ and R5′ comprises a moderate electron-donating group.

10. The N-substituted amide compound of claim 8, wherein said moderate electron-donating group comprises methyl (—CH₃), ethyl (—CH₂—CH₃), propyl (—CH₂—CH₂—CH₃), and isopropyl (—CH₂(CH₃)₃).

11. The N-substituted amide compound of any of claims 1, 2 or 4, wherein J1 is a peptide or polypeptide having one or more optionally protected amino acid side chains, or is a moiety of such a peptide or polypeptide.

12. The N-substituted amide compound of any of claims 1, 2 or 4, wherein J1 is a polymer.

13. The N-substituted amide compound of any of claims 1, 2 or 4, wherein J1 is a dye.

14. The N-substituted amide compound of any of claims 1, 2 or 4, wherein J1 is a functionalized surface.

15. The N-substituted amide compound of any of claims 1, 2 or 4, wherein J1 is a linker or detectable marker.

16. The N-substituted amide compound of any of claims 1, 2 or 4, wherein J2 is a peptide or polypeptide having one or more optionally protected amino acid side chains, or a moiety of such peptide or polypeptide.

17. The N-substituted amide compound of any of claims 1, 2 or 4, wherein J2 is a polymer, a dye, a functionalized surface, a linker or detectable marker; or any other chemical moiety compatible with chemical peptide synthesis or extended native chemical ligation.

18. An acid stable N-substituted 2 or 3 carbon chain amino alkyl or aryl thiol compound of the formula:
HS—C2-C1(R1)-HN-J2 II
or
HS—C3(R3)-C2(R2)-C1(R1)-HN-J2 IV
where:
J1 and J2 are independently a peptide or polypeptide having one or more optionally protected amino acid side chains, or a moiety of such peptide or polypeptide, a polymer, a dye, a functionalized surface, a linker or detectable marker, or, any other chemical moiety compatible with chemical peptide synthesis or extended native chemical ligation; and
R1, R2 and R3 are independently H or an electron donating group conjugated to C1; with the proviso that at least one of R1, R2 and R3 comprises an electron donating group conjugated to C1.

19. The acid stable N-substituted compound of claim 18, wherein said compound has the formula III.

20. The acid stable N-substituted compound of claim 19, wherein C1(R1) is selected from the group consisting of A, B and C: 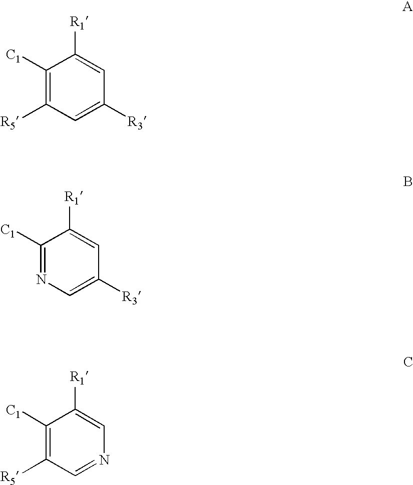
where one or more of R1′, R3′, and R5′ comprise an electron-donating group that may be the same or different.

21. The acid stable N-substituted compound of claim 18, wherein said compound has said formula IV.

22. The acid stable N-substituted compound of claim 21, wherein C3(R3)-C2(R2)-C1(R1) is selected from the group consisting of D, E, F, G, H and I: 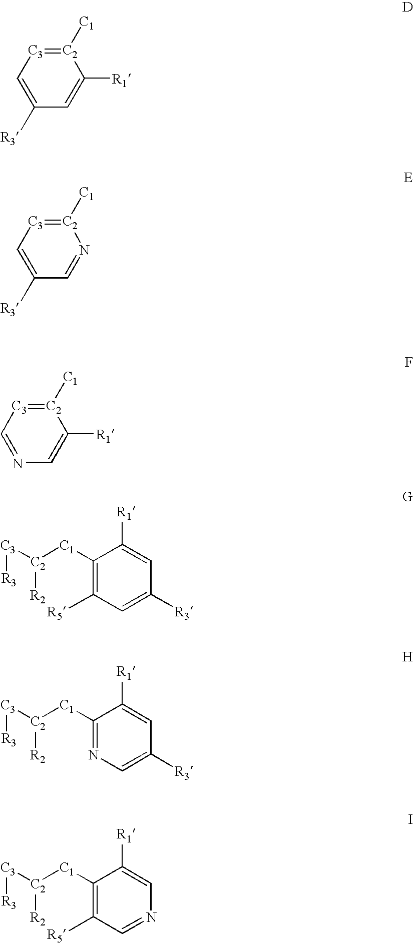
where one or more of R1′, R3′, and R5′ comprise an electron-donating group that may be the same or different.

23. The acid stable N-substituted compound of any of claims 18-22, wherein said substituted N is an Nα-substituted compound.

24. The acid stable N-substituted compound of any of claims 20 or 22, wherein at least one of R1′, R3′ and R5′ comprises a strong electron-donating group.

25. The acid stable N-substituted compound of claim 24, wherein said strong electron-donating group is selected from the group consisting of methoxy (—OCH₃), thiol (—SH), hydroxyl (—OH), and thiomethyl (—SCH₃).

26. The acid stable N-substituted compound of any of claims 20 or 22, wherein at least one of R1′, R3′ and R5′ comprises a moderate electron-donating group.

27. The acid stable N-substituted compound of claim 26, wherein said moderate electron-donating group comprises methyl (—CH₃), ethyl (—CH₂—CH₃), propyl (—CH₂—CH₂—CH₃), and isopropyl (—CH₂(CH₃)₃).

28. The acid stable N-substituted compound of any of claims 18, 19 or 21, wherein J2 is a peptide or polypeptide having one or more optionally protected amino acid side chains, or a moiety of such peptide or polypeptide.

29. The acid stable N-substituted compound of any of claims 18, 19, or 21, wherein J2 is a polymer, a dye, a functionalized surface, a linker or detectable marker; or any other chemical moiety compatible with chemical peptide synthesis or extended native chemical ligation.

30. An N-substituted amide compound of the formula:
J1-C(O)—N(C1(R1)-C2-SH)-J2 I
or
J1-C(O)—N(C1(R1)-C2(R2)-C3(R3)-SH)-J2 II
where:
J1 and J2 are independently a peptide or polypeptide having one or more optionally protected amino acid side chains, or a moiety of such peptide or polypeptide, a polymer, a dye, a functionalized surface, a linker or detectable marker, or, any other chemical moiety compatible with chemical peptide synthesis or extended native chemical ligation; and
R1, R2 and R3 are independently H or an electron donating group conjugated to C1; with the proviso that at least one of said R1, R2 and R3 comprises said electron donating group conjugated to C1;
produced by the process of ligating a first component comprising an α-carboxyl thioester of the formula J1-C(O)SR to a second component comprising an acid stable N-substituted 2 or 3 carbon chain amino alkyl or aryl thiol of the formula:
HS—C2-C1(R1)-HN-J2 III
or
HS—C3(R3)-C2(R2)-C1(R1)-HN-J2 IV
where:
R1, R2 and R3 are independently H or an electron donating group conjugated to C1; with the proviso that at least one of R1, R2 and R3 comprises an electron donating group conjugated to C1.

31. The N-substituted amide compound of claim 30, wherein said acid stable N-substituted compound has the formula III.

32. The N-substituted amide compound of claim 31, wherein C1(R1) of said acid stable N-substituted compound is selected from the group consisting of A, B and C: 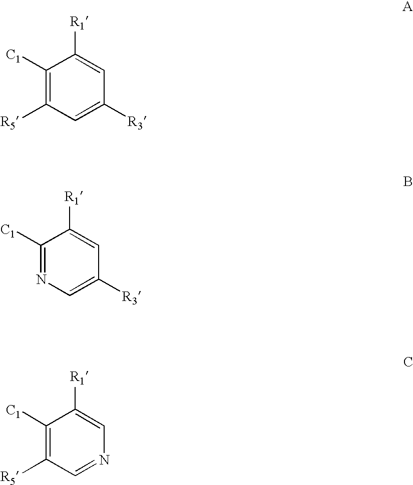
where one or more of R1′, R3′, and R5′ comprise an electron-donating group that may be the same or different.

33. The N-substituted amide compound of claim 30, wherein said compound has said formula IV.

34. The N-substituted amide compound of claim 33, wherein C1(R1)-C2(R2)-C3(R3) is selected from the group consisting of D, E, F, G, H and I: 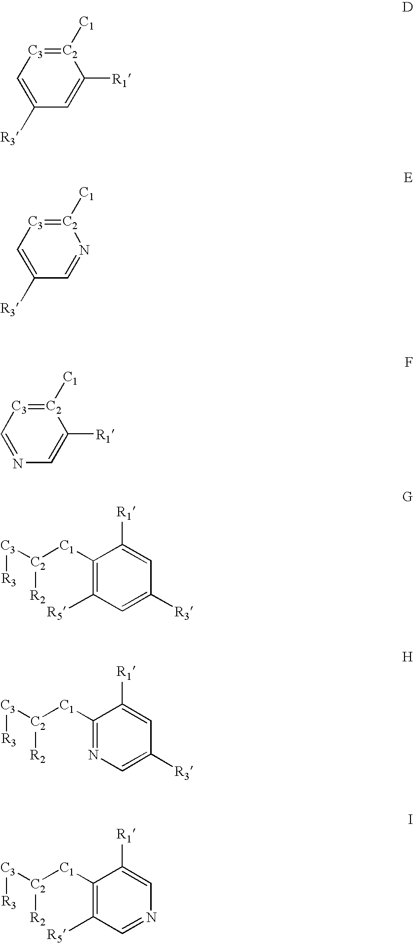
where one or more of R1′, R3′, and R5′ comprise an electron-donating group that may be the same or different.

35. The N-substituted amide compound of any of claims 30-34, wherein said substituted N is an Nα-substituted compound.

36. The N-substituted amide compound of any of claims 32 or 34, wherein at least one of R1′, R3′ and R5′ comprises a strong electron-donating group.

37. The N-substituted amide compound of claim 36, wherein said strong electron-donating group is selected from the group consisting of methoxy (—OCH₃), thiol (—SH), hydroxyl (—OH), and thiomethyl (—SCH₃).

38. The N N-substituted amide compound of any of claims 32 or 34, wherein at least one of R1′, R3′ and R5′ comprises a moderate electron-donating group.

39. The N-substituted amide compound of claim 38, wherein said moderate electron-donating group comprises methyl (—CH₃), ethyl (—CH₂—CH₃), propyl (—CH₂—CH₂—CH₃), and isopropyl (—CH₂(CH₃)₃).

40. The N N-substituted amide compound of any of claims 30, 31 or 33, wherein J1 is a peptide or polypeptide having one or more optionally protected amino acid side chains, or a moiety of such peptide or polypeptide.

41. The N-substituted amide compound of any of claims 30, 31 or 33, wherein J1 is a polymer.

42. The N-substituted amide compound of any of claims 30, 31 or 33, wherein J1 is a dye.

43. The N-substituted amide compound of any of claims 30, 31 or 33, wherein J1 is a functionalized surface.

44. The N-substituted amide compound of any of claims 30, 31 or 33, wherein J1 is a linker or detectable marker.

45. The N-substituted amide compound of any of claims 30, 31 or 33, wherein J2 is a peptide or polypeptide having one or more optionally protected amino acid side chains, or a moiety of such peptide or polypeptide.

46. The N-substituted amide compound of any of claims 30, 31, or 33, wherein J2 is a polymer, a dye, a functionalized surface, a linker or detectable marker; or any other chemical moiety compatible with chemical peptide synthesis or extended native chemical ligation.

47. A compound of the formula:
J1-C(O)—HN-J2 V
where:
J1 and J2 are independently a peptide or polypeptide having one or more optionally protected amino acid side chains, or a moiety of such peptide or polypeptide, a polymer, a dye, a functionalized surface, a linker or detectable marker, or, any other chemical moiety compatible with chemical peptide synthesis or extended native chemical ligation;
wherein said compound is produced by the process of:
(A) ligating a first component comprising an α-carboxyl thioester of the formula J1-C(O)SR to a second component comprising an acid stable N-substituted 2 or 3 carbon chain amino alkyl or aryl thiol of the formula:
HS—C2-C1(R1)-HN-J2 III
where:
R1 is an electron donating group conjugated to C1
to thereby form an N-substituted amide-linked ligation product of the formula:
J1-C(O)—N(C1(R1)-C2-SH)-J2 I
and
(B) removing the 2 carbon chain alkyl or aryl thiol from said N-substituted amide-linked ligation product by cleaving the N—C1 bond.

48. A compound of the formula:
J1-C(O)—HN-J2 V
where:
J1 and J2 are independently a peptide or polypeptide having one or more optionally protected amino acid side chains, or a moiety of such peptide or polypeptide, a polymer, a dye, a functionalized surface, a linker or detectable marker, or, any other chemical moiety compatible with chemical peptide synthesis or extended native chemical ligation;
wherein said compound is produced by the process of:
(A) ligating a first component comprising an α-carboxyl thioester of the formula J1-C(O)SR to a second component comprising an acid stable N-substituted 2 or 3 carbon chain amino alkyl or aryl thiol of the formula:
HS—C3(R3)-C2(R2)-C1(R1)-HN-J2 IV
where
R1, R2 and R3 are independently H or an electron donating group conjugated to C1; with the proviso that at least one of R1, R2 and R3 comprises an electron donating group conjugated to C1;
to thereby form an N-substituted amide-linked ligation product of the formula:
J1-C(O)—N(C1(R1)-C2(R2)-C3(R3)-SH)-J2 II
and
(B) removing the 3 carbon chain alkyl or aryl thiol from said N-substituted amide-linked ligation product by cleaving the N—C1 bond.

49. The compound of claim 47, wherein C1(R1) of said acid stable N-substituted compound is selected from the group consisting of A, B and C: 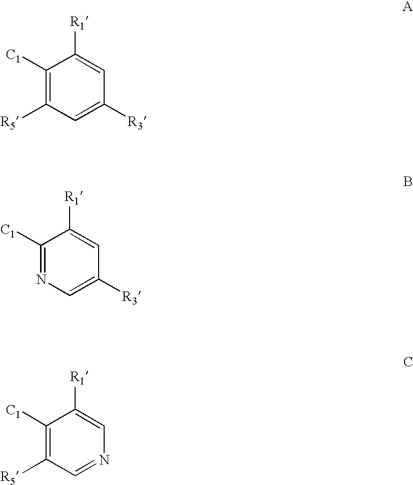
where one or more of R1′, R3′, and R5′ comprise an electron-donating group that may be the same or different.

50. The compound of claim 48, wherein C3(R3)-C2(R2)-C1(R1) of said acid stable N-substituted compound is selected from the group consisting of D, E, F, G, H and I: 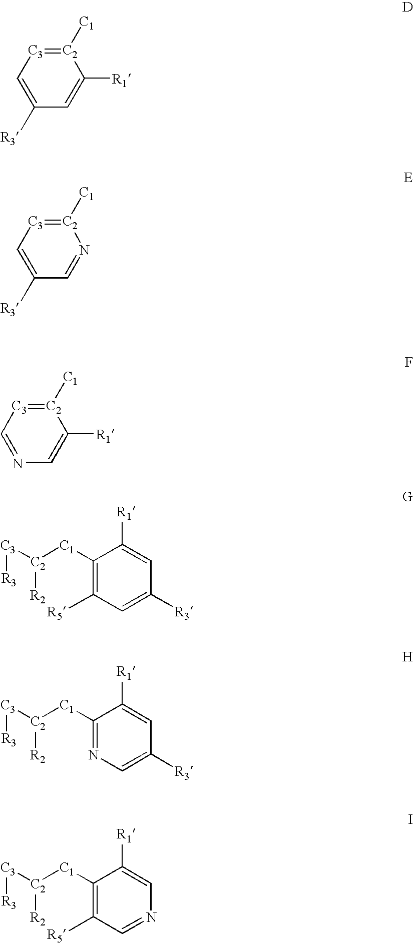
where one or more of R1′, R3′, and R5′ comprise an electron-donating group that may be the same or different.

51. The acid stable N-substituted compound of any of claims 47-50, wherein said N-substituted compound is an Nα-substituted compound.

52. The compound of any of claims 49 or 50, wherein at least one of R1′, R3′ and R5′ comprises a strong electron-donating group.

53. The compound of claim 52, wherein said strong electron-donating group is selected from the group consisting of methoxy (—OCH₃), thiol (—SH), hydroxyl (—OH), and thiomethyl (—SCH₃).

54. The compound of any of claims 49 or 50, wherein at least one of R1′, R3′ and R5′ comprises a moderate electron-donating group.

55. The compound of claim 54, wherein said moderate electron-donating group comprises methyl (—CH₃), ethyl (—CH₂—CH₃), propyl (—CH₂—CH₂—CH₃), and isopropyl (—CH₂(CH₃)₃).

56. The compound of any of claims 47 or 48, wherein J1 is a peptide or polypeptide having one or more optionally protected amino acid side chains.

57. The compound of any of claims 47 or 48, wherein J1 is a polypeptide having one or more optionally protected amino acid side chains.

58. The compound of any of claims 47 or 48, wherein J1 is a polymer.

59. The compound of any of claims 47 or 48, wherein J1 is a dye.

60. The compound of any of claims 47 or 48, wherein J1 is a functionalized surface.

61. The compound of any of claims 47 or 48, wherein J1 is a linker or detectable marker.

62. The compound of any of claims 47 or 48, wherein J2 is a peptide or polypeptide having one or more optionally protected amino acid side chains, or a moiety of such peptide or polypeptide.

63. The compound of any of claims 47 or 48, wherein J2 is a polymer, a dye, a functionalized surface, a linker or detectable marker; or any other chemical moiety compatible with chemical peptide synthesis or extended native chemical ligation.

64. A method for producing a compound of the formula:
J1-C(O)—HN-J2 V
where:
J1 and J2 are independently a peptide or polypeptide having one or more optionally protected amino acid side chains, or a moiety of such peptide or polypeptide, a polymer, a dye, a functionalized surface, a linker or detectable marker, or, any other chemical moiety compatible with chemical peptide synthesis or extended native chemical ligation;
wherein said method comprises the steps of:
(A) ligating a first component comprising an a-carboxyl thioester of the formula J1-C(O)SR to a second component comprising an acid stable N-substituted 2 or 3 carbon chain amino alkyl or aryl thiol of the formula:
HS—C2-C1(R1)-HN-J2 III
where:
R1 is an electron donating group conjugated to C1
to thereby form an N-substituted amide-linked ligation product of the formula:
J1-C(O)—N(C1(R1)-C2-SH)-J2 I
(B) removing the 2 carbon chain alkyl or aryl thiol from said N-substituted amide-linked ligation product by cleaving the Nα-C1 bond.

65. A method for producing a compound of the formula:
J1-C(O)—HN-J2 V
where:
J1 and J2 are independently a peptide or polypeptide having one or more optionally protected amino acid side chains, or a moiety of such peptide or polypeptide, a polymer, a dye, a functionalized surface, a linker or detectable marker, or, any other chemical moiety compatible with chemical peptide synthesis or extended native chemical ligation;
wherein said method comprises the steps of:
(A) ligating a first component comprising an α-carboxyl thioester of the formula J1-C(O)SR to a second component comprising an acid stable N-substituted 2 or 3 carbon chain amino alkyl or aryl thiol of the formula:
HS—C3(R3)-C2(R2)-C1(R1)-HN-J2 IV
where
R1, R2 and R3 are independently H or an electron donating group conjugated to C1; with the proviso that at least one of R1, R2 and R3 comprises an electron donating group conjugated to C1;
to thereby form an N-substituted amide-linked ligation product of the formula:
J1-C(O)—N(C1(R1)-C2(R2)-C3(R3)-SH)-J2 II
(B) removing the 3 carbon chain alkyl or aryl thiol from said N-substituted amide-linked ligation product by cleaving the Nα-C1 bond.

66. The method of claim 64, wherein C1(R1) of said acid stable N-substituted compound is selected from the group consisting of A, B and C: 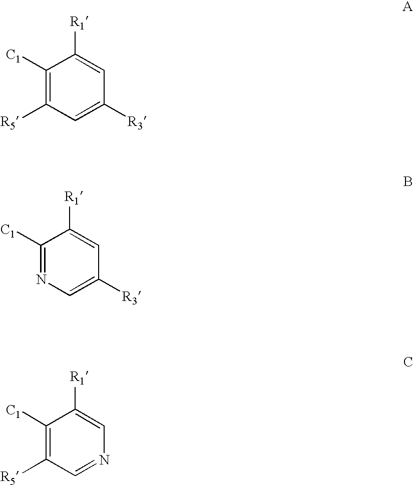
where one or more of R1′, R3′, and R5′ comprise an electron-donating group that may be the same or different.

67. The method of claim 65, wherein C1(R1)-C2(R2)-C3(R3) of said acid stable N-substituted compound is selected from the group consisting of D, E, F, G, H and I: 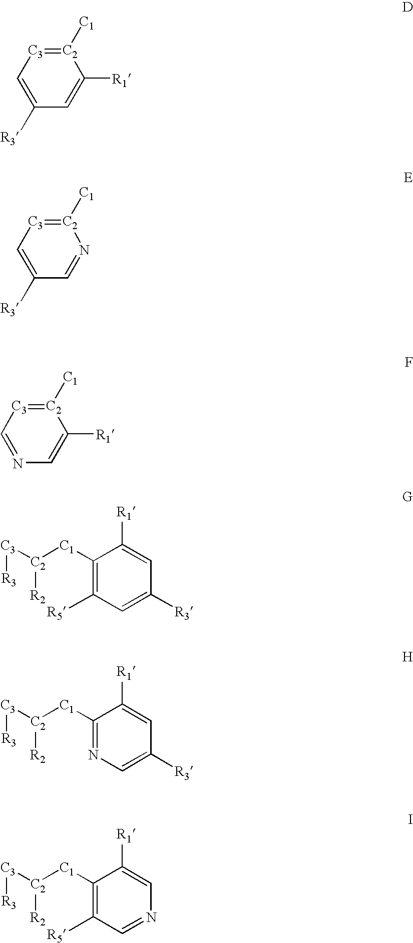
where one or more of R1′, R3′, and R5′ comprise an electron-donating group that may be the same or different.

68. The method of any of claims 64-67, wherein said N-substituted compound is an Nα-substituted compound.

69. The method of any of claims 66 or 67, wherein at least one of R1′, R3′ and R5′ comprises a strong electron-donating group.

70. The method of claim 66, wherein said strong electron-donating group of said N-substituted compound is selected from the group consisting of methoxy (—OCH₃), thiol (—SH), hydroxyl (—OH), and thiomethyl (—SCH₃).

71. The method of any of claims 66 or 67, wherein at least one of R1′, R3′ and R5′ of said N-substituted compound comprises a moderate electron-donating group.

72. The method of claim 71, wherein said moderate electron-donating group of said N-substituted compound comprises methyl (—CH₃), ethyl (—CH₂—CH₃), propyl (—CH₂—CH₂—CH₃), and isopropyl (—CH₂(CH₃)₃).

73. The method of any of claims 66 or 67, wherein J1 is a peptide or polypeptide having one or more optionally protected amino acid side chains, or a moiety of such peptide or polypeptide.

74. The method of any of claims 66 or 67, wherein J1 is a polymer.

75. The method of any of claims 66 or 67, wherein J1 is a dye.

76. The method of any of claims 66 or 67, wherein J1 is a functionalized surface.

77. The method of any of claims 66 or 67, wherein J1 is a linker or detectable marker.

78. The method of any of claims 66 or 67, wherein J2 is a peptide or polypeptide having one or more optionally protected amino acid side chains, or a moiety of such peptide or polypeptide.

79. The method of any of claims 66 or 67, wherein J2 is a polymer, a dye, a functionalized surface, a linker or detectable marker; or any other chemical moiety compatible with chemical peptide synthesis or extended native chemical ligation.

80. The method of any of claims 66 or 67, wherein said compound is synthesized in solution.

81. The method of any of claims 66 or 67, wherein said compound is synthesized immobilized to a solid support.


SEQUENCE LISTING (1 sequence)

SEQ ID NO: 1 - protein, 106 aa, Homo sapiens
  PEPTIDE (1)..(106)
ADLEDNMETLNDNLKVIEKADNAAQVKDALTKMRAAALDAQKATPPKLEDKSPDSPEMKDFRHGFDILVGQIDDALKLANEGKVKEAQAAAEQLKTTRNAYHQKYR